(19) United States
(12) Patent Application Publication
(10) Pub. No.: US 2024/0336903 A1
(43) Pub. Date: Oct. 10, 2024

(54) Fusion Proteins for Dephosphorylating Proteins that Regulate T Cell Activation through the TCR Signaling Pathway

(71) Applicant: Shandong Boan Biotechnology Co., Ltd., Shandong (CN)

(72) Inventors: Michael Harris, Woburn, MA (US); Ninghai Wang, Woburn, MA (US); Li Zhou, Woburn, MA (US); Feng Shi, Woburn, MA (US)

(21) Appl. No.: 18/579,836

(22) PCT Filed: Aug. 18, 2022
(86) PCT No.: PCT/US2022/075150

§ 371 Date: Jan. 16, 2024

Related U.S. Application Data
(60) Provisional application No. 63/290,576, filed on Dec. 16, 2021.
(60) Provisional application No. 63/235,119, filed on Aug. 19, 2021.

(51) Int. Cl. C12N 9/16; A61K 38/00; C07K 14/705; C07K 14/725; C07K 16/30; C12N 5/0783

(52) U.S. Cl. CPC C12N 9/16; C07K 14/7051; C07K 14/70589; C07K 14/70596; C07K 16/3007; C12N 5/0636; A61K 38/00; C07K 2317/622; C07K 2319/02; C07K 2319/03; C12Y 301/03048

(57) ABSTRACT

The present application provides novel fusion proteins for dephosphorylating proteins that regulate T cell activation through the TCR signaling pathway, nucleic acids encoding said proteins, vectors comprising said nucleic acids, compositions comprising said nucleic acids or vectors, host cells comprising said nucleic acids, vectors or compositions or related pharmaceutical compositions. The present application also provides methods for dephosphorylation of proteins that regulate T cell activation through the TCR signaling pathway of a cell, methods of producing a cell having dephosphorylated proteins that regulate T cell activation through the TCR signaling pathway (e.g. a CAR T cell having dephosphorylated Immunoreceptor Tyrosine-based Activation Motifs (ITAMs) of endogenous CD3), methods of treating a disease, and methods of reducing or preventing GvHD in a subject associated with the administration of one or more CAR T-cells to the subject.

This application is a US National Phase of PCT Application No. PCT/US2022/075150, filed on Aug. 18, 2022, which claims the benefit of U.S. Provisional Patent Application No. 63/235,119, entitled “Fusion Proteins for Dephosphorylating Proteins that Regulate T Cell Activation through the TCR Signaling Pathway”, filed on Aug. 19, 2021, and U.S. Provisional Patent Application No. 63/290,576, entitled “Fusion Proteins for Dephosphorylating Proteins that Regulate T Cell Activation through the TCR Signaling Pathway” filed on Dec. 16, 2021; the contents of which are herein incorporated by reference in their entireties.

The subject matter disclosed herein and the presently claimed invention was made by or on behalf of the below listed parties to a joint research agreement. The joint research agreement was in effect on or before the effective filing date of the claimed invention, and the claimed invention was made as a result of activities undertaken within the scope of the joint research agreement. The parties to the joint research agreement are Shandong Boan Biotechnology Co., Ltd. and Boan Boston LLC.

### TECHNICAL FIELD

This application relates to novel fusion proteins for dephosphorylating proteins that regulate T cell activation through the TCR signaling pathway, nucleic acids encoding said fusion proteins, vectors comprising said nucleic acids, compositions comprising said nucleic acids or vectors, host cells comprising said nucleic acids, vectors or compositions or related pharmaceutical compositions. The present application also provides methods for dephosphorylating proteins that regulate T-cell activation through the TCR signaling pathway (e.g. proteins that rely on phosphorylation as an activation mechanism for signaling in the TCR signaling pathway), wherein the proteins that regulate T-cell activation through the TCR signaling pathway may comprise one or more subunits or structural domains of membrane-bound proteins (MBPs) of the T cell, kinases and/or scaffold proteins; methods of producing a T cell having dephosphorylated subunits or structural domains of membrane-bound proteins (MBPs) in the TCR signaling pathway (e.g. a CAR T cell having dephosphorylated Immunoreceptor Tyrosine-based Activation Motifs (ITAMs) of CD3 in the TCR signaling pathway; optionally, dephosphorylated ITAMs of endogenous CD3); methods of treating a disease; or methods of reducing or preventing GvHD in a subject associated with the administration of one or more of the CAR T-cells to the subject.

### BACKGROUND

Chimeric Antigen Receptor (CAR) T cell therapy has so far been successful in the treatment of several types of hematological malignancies and as the field progresses, it is expected that we will see more applications for this type of immunotherapy in the treatment of a more diverse array of cancers. CARs are a type of synthetic receptor designed to mimic the activity of the T cell antigen receptor (TCR). They are typically comprised of an extracellular antigen recognition motif, which in most formats is derived from an antibody binding domain (scFv), and the ITAM signaling is motifs of CD3ζ. More recent CAR formats, including those in clinical use, also have a T cell co-receptor signaling domain, such as those derived from CD28 or 4-1BB. As the disease applications for CARs become broader, so too do the structural variants of CARs, increasing the diversity of CAR formats.

Perhaps the largest roadblock in the adoption of CAR T cells as a more routine form of immunotherapy is the lack of standardized, “off-the-shelf” treatment formats. All of the CAR formats currently approved for clinical use are autologous treatments that must be generated on a per-patient basis, drastically increasing the cost of CAR T cell therapy and resulting in a high degree of patient-to-patient variability, which ultimately influences treatment outcome. Generation of a reliable allogeneic CAR T cell format could alleviate many of the challenges encountered in the field of CAR T cells. Allogeneic CAR T cells would allow for repeatable, titratable dosing to reduce toxicity and could be generated in alternative, standardized formats to address antigen escape. Selection of specific allogeneic donors could also be important in the generation of desirable or unique activation profiles in response to target antigen.

Given the importance of allogeneic T cells to the establishment of CARs as a frontline cancer therapy, several methods have previously been described to generate allogeneic CAR T cells. These primarily involve modification of the donor cells at the chromosomal level, including non-homologous recombination of the TRAC locus to ablate TCRα while simultaneously knocking-in CAR expression. Other methods include CRISPR-Cas9 or TALEN-mediated disruption of the constant regions of the TCR locus. Although genetic manipulation of the TCR locus is an attractive and often effective way of preventing graft-vs-host recognition, engineering efficiency can often be low and the on-target chromothripsis that occurs due to CRISPR-Cas9 gene-editing has as-yet unknown consequences that will likely be revealed as the latest generation of gene therapies progresses through the clinic. Others have generated allogeneic CAR T cells through the repurposing of virus-specific T cells compatible with a large percentage of hosts.

Graft-vs-host disease occurs when T cells derived from an allogeneic donor recognize non-self pMHC of the engrafted host. The recognition of non-self peptide ligands by the donor T cells results in targeted destruction of host tissues and organs and can trigger a potentially fatal inflammatory response. This T cell-target cell recognition occurs due to imperfect HLA matching and is the reason that many hematopoietic stem cell recipients must be maintained in a state of chronic immunosuppression.

At the most fundamental level, TCR triggering occurs when the TCR antigen receptor complex encounters cognate peptide-bound Major Histocompatibility Complex (pMHC). Recognition of pMHC by the TCR ultimately results in receptor clustering and eventually the reorganization of signaling molecules at the T cell-target cell membrane interface into a structure termed the ‘immunological synapse’. The formation of TCR receptor microclusters is crucial for complete T cell activation. These microclusters form at regions of tight membrane apposition between the T cell and the target cell and result in the exclusion of proteins with large, rigid extracellular domains, such as CD45. This observation has given rise to the Kinetic Segregation model of T cell activation, which states that during receptor clustering, kinases such as Lck and ZAP70 are able to access the ITAM motifs of the TCR, resulting in phosphorylation and the propagation of downstream signaling. However, these same ITAM motifs are inaccessible to phosphatases, such as CD45 or CD148, due to their large extracellular domains.

A major problem in the field of CAR T cell therapy that must be solved is the development of allogeneic technologies that specifically inactivate the TCR without broadly inactivating the CAR at the same time. Using chimeric phosphatases, we have designed a way to specifically inactivate TCR signaling without genetically knocking out, degrading, or knocking down expression of components of the TCR.

### SUMMARY OF THE INVENTION

The present application provides novel fusion proteins (i.e., chimeric phosphatases) for dephosphorylating proteins that regulate T cell activation (e.g. through the TCR signaling pathway), nucleic acids encoding said fusion proteins, fusion protein expression cassettes comprising said nucleic acids, vectors comprising said nucleic acids, compositions comprising said nucleic acids or vectors, host cells comprising said nucleic acids, vectors or compositions or related pharmaceutical compositions. The present application also provides methods for dephosphorylating proteins that regulate T-cell activation (e.g., proteins that rely on phosphorylation as an activation mechanism for signaling in the TCR signaling pathway). The proteins that regulate T cell activation through the TCR signaling pathway may comprise one or more subunits or structural domains of a protein selected from one or more of a membrane-bound protein (MBP), a kinase and a scaffold protein of the T cell. The present application also provides methods of producing a T cell having dephosphorylated subunits or structural domains of a membrane-bound protein (MBP) in the TCR signaling pathway (e.g. a CAR T cell having dephosphorylated Immunoreceptor Tyrosine-based Activation Motifs (ITAMs) of MBPs; optionally, dephosphorylated ITAMs of endogenous MBPs), methods of treating a disease, or methods of reducing or preventing GvHD in a subject associated with the administration of one or more of the CAR T-cells to the subject.

In the present application, the dephosphorylation of proteins that regulate T cell activation through the TCR signaling pathway (wherein preferably, the proteins rely on phosphorylation as an activation mechanism for signaling in the TCR signaling pathway, such as one or more subunits or structural domains of a protein selected from one or more of a membrane-bound protein (MBP), a kinase and a scaffold protein of the T cell) is achieved by a dephosphorylation protein, wherein the dephosphorylation protein comprises one or more subunits or structural domains of phosphatases (e.g. receptor-like protein tyrosine phosphatases, preferably, CD45 or CD148) or variants thereof. The targeting specificity of the dephosphorylation protein for the proteins that regulate T cell activation through the TCR signaling pathway is mediated by a linking protein (e.g. the transmembrane domain of one or more subunits of a membrane-bound protein (MBP)). T cell activation through the TCR signaling pathway thus can be regulated by the fusion proteins without degrading, knocking down, or genetically knocking out the components of the TCR signaling pathway. In allogeneic CAR T cells, these fusion proteins specifically inactivate TCR signaling, but do not broadly inactivate both the TCR and CAR signaling. This can be achieved without degrading, knocking down, or genetically knocking out the components of TCR signaling; and so Graft vs Host Disease (GvHD) in a subject associated with the administration of the CAR T-cells can be reduced or prevented.

In one aspect, the fusion protein comprises: a dephosphorylation protein and a linking protein, wherein
the dephosphorylation protein comprises one or more subunits or structural domains of phosphatases or variants thereof, and
the linking protein comprises a transmembrane domain of one or more subunits of a membrane-bound protein (MBP) of a T cell, or the linking protein comprises an extracellular domain and a transmembrane domain of one or more subunits of a membrane-bound protein (MBP) of a T cell.

In a specific embodiment, as the linking protein, the MBP comprises one or more of TCR and CD3; preferably, the MBP is a mammalian origin MBP, more preferably a human MBP; and more preferably, the extracellular domain is derived from an extracellular domain of one or more of CD3ζ, CD3γ, CD3δ, CD3ε, TCRα or TCRβ;
more preferably, the transmembrane domain is derived from a transmembrane domain of one or more of CD3ζ, CD3γ, CD3δ, CD3ε, TCRα or TCRβ.

In a specific embodiment, the phosphatases are receptor-like protein tyrosine phosphatases; preferably, the dephosphorylation protein comprises an intracellular phosphatase domain of the receptor-like protein tyrosine phosphatases or a variant thereof; more preferably, the receptor-like protein tyrosine phosphatases comprise CD45 or CD148.

In a specific embodiment, the fusion protein comprises, from N-terminal to C-terminal: (1) the linking protein; and (2) the dephosphorylation protein.

In a specific embodiment, the fusion protein comprises, from N-terminal to C-terminal: (1) a transmembrane domain of one or more subunits of membrane-bound protein (MBP) of a T cell; and (2) the dephosphorylation protein.

In a specific embodiment, the fusion protein comprises, from N-terminal to C-terminal: (1) an extracellular domain, a transmembrane domain of one or more subunits of membrane-bound protein (MBP) of a T cell; and (2) the dephosphorylation protein.

In a specific embodiment, the fusion protein comprises, from N-terminal to C-terminal: (1) the transmembrane domain of one or more of CD3ζ, CD3γ, CD3δ, CD3ε, TCRα or TCRβ or variants thereof, and (2) the intracellular phosphatase domain of CD45 or CD148 or a variant thereof.

In a specific embodiment, the fusion protein comprises, from N-terminal to C-terminal: (1) the extracellular domain of one or more of CD3ζ, CD3γ, CD3δ, CD3ε, TCRα or TCRβ or variants thereof, the transmembrane domain of one or more of CD3ζ, CD3γ, CD3δ, CD3ε, TCRα or TCRβ or variants thereof, and (2) the intracellular phosphatase domain of CD45 or CD148 or a variant thereof.

In a specific embodiment, the fusion protein comprises, from N-terminal to C-terminal: (1) the transmembrane domain of one or more of CD3ζ, CD3γ, CD3δ, CD3ε, or variants thereof, and (2) the intracellular phosphatase domain of CD45 or CD148 or a variant thereof.

In a specific embodiment, the fusion protein comprises, from N-terminal to C-terminal: (1) the extracellular domain of one or more of CD3ζ, CD3γ, CD3δ, CD3ε, or variants thereof, the transmembrane domain of one or more of CD3ζ, CD3γ, CD3δ, CD3ε, or variants thereof, and (2) the intracellular phosphatase domain of CD45 or CD148 or a variant thereof.

In a specific embodiment, the fusion protein comprises, from N-terminal to C-terminal: (1) the extracellular domain of one or more of CD3ζ or variants thereof, the transmembrane domain of one or more of CD3ζ or variants thereof, and (2) the intracellular phosphatase domain of CD45 or a variant thereof.

In a specific embodiment, the fusion protein comprises, from N-terminal to C-terminal: (1) the extracellular domain of one or more of CD3ζ or variants thereof, the transmembrane domain of one or more of CD3ζ or variants thereof, and (2) the intracellular phosphatase domain of CD148 or a variant thereof.

In a specific embodiment, the fusion protein comprises, from N-terminal to C-terminal: (1) the extracellular domain of one or more of CD3ε or variants thereof, the transmembrane domain of one or more of CD3ε or variants thereof, and (2) the intracellular phosphatase domain of CD148 or a variant thereof.

In a specific embodiment, the fusion protein comprises, from N-terminal to C-terminal: (1) the extracellular domain of one or more of CD3δ or variants thereof, the transmembrane domain of one or more of CD3δ or variants thereof, and (2) the intracellular phosphatase domain of CD148 or a variant thereof.

In a specific embodiment, the fusion protein comprises, from N-terminal to C-terminal: (1) the extracellular domain of one or more of CD3γ or variants thereof, the transmembrane domain of one or more of CD3γ or variants thereof, and (2) the intracellular phosphatase domain of CD148 or a variant thereof.

In a specific embodiment, the fusion protein comprises, from N-terminal to C-terminal: (1) the extracellular domain of one or more of CD3ζ or variants thereof, the transmembrane domain of one or more of CD3ζ or variants thereof, (2) the intracellular phosphatase domain of CD148 or a variant thereof, and (3) a puromycin selection marker and/or GFP.

In a specific embodiment, the fusion protein comprises, from N-terminal to C-terminal: (1) the extracellular domain of one or more of CD3ε or variants thereof, the transmembrane domain of one or more of CD3ε or variants thereof, (2) the intracellular phosphatase domain of CD148 or a variant thereof, and (3) a puromycin selection marker and/or GFP.

In a specific embodiment, the fusion protein comprises, from N-terminal to C-terminal: (1) the extracellular domain of one or more of CD3δ or variants thereof, the transmembrane domain of one or more of CD3δ or variants thereof, (2) the intracellular phosphatase domain of CD148 or a variant thereof, and (3) a puromycin selection marker and/or GFP.

In a specific embodiment, the fusion protein comprises, from N-terminal to C-terminal: (1) the extracellular domain of one or more of CD3γ or variants thereof, the transmembrane domain of one or more of CD3γ or variants thereof, (2) the intracellular phosphatase domain of CD148 or a variant thereof, and (3) a puromycin selection marker and/or GFP.

In a specific embodiment, the fusion protein is capable of mediating dephosphorylation of proteins that regulate T cell activation (e.g. through the TCR signaling pathway), and the proteins that regulate T cell activation comprise one or more subunits or structural domains of a protein selected from one or more of a membrane-bound protein (MBP), a kinase, or a scaffold protein of the T cell;
preferably, the proteins that regulate T cell activation comprise one or more subunits or structural domains of TCR or CD3; more preferably, the TCR or CD3 is a mammalian origin TCR or CD3, more preferably a human TCR or CD3; and more preferably an endogenous TCR or CD3; more preferably, the proteins that regulate T cell activation comprise ITAM domains of the membrane-bound protein (MBP), preferably ITAM domains of CD3, more preferably ITAM domains of CD3 ζ;
preferably, the kinase comprises ZAP70; and more preferably, the kinase is a mammalian origin kinase, more preferably a human kinase; or
preferably, the scaffold protein comprises LAT and/or SLP76; and more preferably, the scaffold protein is a mammalian origin scaffold protein, more preferably a human scaffold protein.

In a further embodiment, the transmembrane domain of CD3ζ comprises an amino acid sequence at least about 95%, 96%, 97%, 98%, 99% or 100% identical to the amino acid sequence shown in SEQ ID NO. 3, the transmembrane domain of CD3ε comprises an amino acid sequence at least about 95%, 96%, 97%, 98%, 99% or 100% identical to the amino acid sequence shown in SEQ ID NO. 28, the transmembrane domain of CD3γ comprises an amino acid sequence at least about 95%, 96%, 97%, 98%, 99% or 100% identical to the amino acid sequence shown in SEQ ID NO. 31, and the transmembrane domain of CD3δ comprises an amino acid sequence at least about 95%, 96%, 97%, 98%, 99% or 100% identical to the amino acid sequence shown in SEQ ID NO. 34.

In a further embodiment, the extracellular domain of CD3ζ comprises an amino acid sequence at least about 95%, 96%, 97%, 98%, 99% or 100% identical to the amino acid sequence shown in SEQ ID NO. 2, the extracellular domain of CD3ε comprises an amino acid sequence at least about 95%, 96%, 97%, 98%, 99% or 100% identical to the amino acid sequence shown in SEQ ID NO. 27, the extracellular domain of CD3γ comprises an amino acid sequence at least about 95%, 96%, 97%, 98%, 99% or 100% identical to the amino acid sequence shown in SEQ ID NO. 30, and the extracellular domain of CD3δ comprises an amino acid sequence at least about 95%, 96%, 97%, 98%, 99% or 100% identical to the amino acid sequence shown in SEQ ID NO. 33.

In a further embodiment, the intracellular phosphatase domain of CD45 comprises an amino acid sequence at least about 95%, 96%, 97%, 98%, 99% or 100% identical to the amino acid sequence shown in SEQ ID NO. 4; and/or the intracellular phosphatase domain of CD148 comprises an amino acid sequence at least about 95%, 96%, 97%, 98%, 99% or 100% identical to the amino acid sequence shown in SEQ ID NO. 5.

In a further embodiment, the fusion protein comprises a signal peptide sequence, wherein the is signal peptide sequence comprises an amino acid sequence at least about 95%, 96%, 97%, 98%, 99% or 100% identical to the amino acid sequence shown in any one of SEQ ID NOs:1, 26, 29, or 32;
preferably, the CD3ζ signal peptide sequence comprises an amino acid sequence at least about 95%, 96%, 97%, 98%, 99% or 100% identical to the amino acid sequence shown in SEQ ID NO. 1, the CD3ε signal peptide sequence comprises an amino acid sequence at least about 95%, 96%, 97%, 98%, 99% or 100% identical to the amino acid sequence shown in SEQ ID NO. 26, the CD3γ signal peptide sequence comprises an amino acid sequence at least about 95%, 96%, 97%, 98%, 99% or 100% identical to the amino acid sequence shown in SEQ ID NO. 29, and the CD3δ signal peptide sequence comprises an amino acid sequence at least about 95%, 96%, 97%, 98%, 99% or 100% identical to the amino acid sequence shown in SEQ ID NO. 32.

In another aspect, the present application provides a nucleic acid comprising a polynucleotide encoding a fusion protein of the present application.

In another aspect, the present application provides a fusion protein expression cassette comprising a nucleic acid encoding both the fusion protein and a purification selection marker; more preferably, the purification selection marker is a puromycin selection marker; more preferably, the puromycin selection marker comprises an amino acid sequence at least about 95%, 96%, 97%, 98%, 99% or 100% identical to the amino acid sequence shown in SEQ ID NO. 25.

In another aspect, the present application provides a vector comprising a nucleic acid encoding the fusion protein, or the fusion protein expression cassette.

In another aspect, the present application provides a composition comprising a nucleic acid or a vector of the present application.

In a specific embodiment, the composition comprises a first nucleic acid and a second nucleic acid, wherein
(1) the first nucleic acid encodes the fusion protein of the application, and
(2) the second nucleic acid encodes a chimeric antigen receptor (CAR) comprising:
(a) an extracellular ligand-binding domain comprising a single chain variable fragment (scFv) specifically binding to a predetermined antigen;
(b) a transmembrane domain, preferably CD8α, CD28, 4-1BB or IL2Rβ transmembrane domain, more preferably CD8α transmembrane domain; and
(c) a cytoplasmic segment comprising one or more signaling domains, preferably comprising a 4-1BB signaling domain and a CD3ζ signaling domain.

The present application further provides a composition comprising a first vector having a first nucleic acid and a second vector having a second nucleic acid, wherein
(1) the first nucleic acid encodes the fusion protein of the application, and
(2) the second nucleic acid encodes a chimeric antigen receptor (CAR) comprising:
(a) an extracellular ligand-binding domain comprising a single chain variable fragment (scFv) specifically binding to a predetermined antigen;
(b) a transmembrane domain, preferably CD8α, CD28, 4-1BB or IL2Rβ transmembrane domain, more preferably CD8α transmembrane domain; and
(c) a cytoplasmic segment comprising one or more signaling domains, preferably comprising a 4-1BB signaling domain and a CD3ζ signaling domain.

The present application further provides a composition comprising a vector having a first nucleic acid and a second nucleic acid, wherein
(1) the first nucleic acid encodes the fusion protein of the application, and
(2) the second nucleic acid encodes a chimeric antigen receptor (CAR) comprising:
(a) an extracellular ligand-binding domain comprising a single chain variable fragment (scFv) specifically binding to a predetermined antigen;
(b) a transmembrane domain, preferably CD8α, CD28, 4-1BB or IL2Rβ transmembrane domain, more preferably CD8α transmembrane domain; and
(c) a cytoplasmic segment comprising one or more signaling domains, preferably comprising a 4-1BB signaling domain and a CD3ζ signaling domain.

In a specific embodiment, the predetermined antigen is a tumor-related antigen.

In a further embodiment, the tumor-related antigen is selected from the following group: CEA, Claudin 18.2 (i.e. CLDN18.2), GPC3, Receptor tyrosine kinase-like Orphan Receptor 1 (ROR1), CD38, CD19, CD20, CD22, BCMA, CAIX, CD446, CD133, EGFR, EGFRvIII, EpCam, GD2, EphA2, Her1, Her2, ICAM-1, IL13Ra2, Mesothelin, MUC1, MUC16, NKG2D, PSCA, NY-ESO-1, MART-1, WT1, MAGE-A10, MAGE-A3, MAGE-A4, EBV, NKG2D, PD1, PD-L1, CD25, IL-2, and CD3.

In a further embodiment, the tumor-related antigen is CEA, more preferably CEACAM5.

In a further embodiment, the chimeric antigen receptor (CAR) encoded by the second nucleic acid comprises, from N-terminal to C-terminal:
MN14op scFv-CD8α hinge-CD8αTM-4-1BB-CD3ζ int;
841 scFv-CD8α hinge-CD8αTM-4-1BB-CD3ζ int; or
CD8α signal peptide -MN14op scFv-CD8α hinge-CD8αTM-4-1BB-CD3ζ int-P2A-GFP.

In a further embodiment, the present application provides an AC7 construct obtained from the vector having a first nucleic acid and a second nucleic acid comprising, from N-terminal to C-terminal:
SP-CD3ζ ex/tm-CD148 int-P2A-SP-CEACAM-5 scFv-CD8α hinge-CD8αTM-4-1BB-CD3ζ int.

In a further embodiment, the N-terminal of the CAR further contains a leader sequence, and the C-terminal of the CAR further contains a P2A-GFP sequence, wherein,
the leader sequence comprises an amino acid sequence that is at least about 95%, 96%, 97%, 98%, 99% or 100% identical to the amino acid sequence represented by SEQ ID NO. 11,
the CD8α Hinge comprises an amino acid sequence that is at least about 95%, 96%, 97%, 98%, 99% or 100% identical to the amino acid sequence represented by SEQ ID NO.: 14,
the CD8α tm comprises an amino acid sequence that is at least about 95%, 96%, 97%, 98%, 99% or 100% identical to the amino acid sequence represented by SEQ ID NO.: 15,
the 4-1BB intracellular domain comprises an amino acid sequence that is at least about 95%, 96%, 97%, 98%, 99% or 100% identical to the amino acid sequence represented by SEQ ID NO.: 16,
the CD3ζ intracellular domain comprises an amino acid sequence that is at least about 95%, 96%, 97%, 98%, 99% or 100% identical to the amino acid sequence represented by SEQ ID NO.: 17,
the GFP comprises an amino acid sequence that is at least about 95%, 96%, 97%, 98%, 99% or 100% identical to the amino acid sequence represented by SEQ ID NO.: 23, and
the P2A comprises an amino acid sequence that is at least about 95%, 96%, 97%, 98%, 99% or 100% identical to the amino acid sequence represented by SEQ ID NO. 22.

In a further embodiment, MN14op scFv (i.e. CEACAM-5 scFv comprises an amino acid sequence that is at least about 95%, 96%, 97%, 98%, 99% or 100% identical to the amino acid sequence represented by SEQ ID NO. 12;
the 841 scFv comprises an amino acid sequence that is at least about 95%, 96%, 97%, 98%, 99% or 100% identical to the amino acid sequence represented by SEQ ID NO. 13.

In another aspect, the present application further provides a host cell, the host cell comprising a nucleic acid or a vector or a composition of the present application.

In a further embodiment, the host cell is a mammalian T cell, preferably a human T cell.

In a further embodiment, the host cell is a Jurkat cell, a primary T cell, a gamma delta T cell, or a NK T cell.

In a further embodiment, the host cell is an allogeneic T cell.

In some embodiments, the host cell is an allogeneic CAR T cell. The allogeneic CAR T cell has dephosphorylated Immunoreceptor Tyrosine-based Activation Motifs (ITAMs) of the endogenous CD3 of the TCR/CD3 complex, and a CAR as mentioned above.

In another aspect, the present application further provides a pharmaceutical composition, the pharmaceutical composition comprising the nucleic acid, vector, composition or host cell of the present application.

In a specific embodiment, the composition further comprises one or more pharmaceutically acceptable excipients.

In another aspect, the present application provides a method for the dephosphorylation of proteins that regulate T cell activation through the TCR signaling pathway, wherein preferably, the proteins rely on phosphorylation as an activation mechanism for signaling in the TCR signaling pathway, preferably, the proteins are Immunoreceptor Tyrosine-based Activation Motifs (ITAMs) of a membrane-bound protein (MBP) in the TCR signaling pathway of a T cell, comprising introducing into a T cell the nucleic acid, the vector, or the composition of the present application.

In a specific embodiment, the T cell is a mammalian T cell, preferably a human T cell.

In a specific embodiment, the T cell is a Jurkat cell, a primary T cell, a gamma delta T cell, or a NKT cell.

In a further embodiment, the T cell is an allogeneic T cell.

In another aspect, the present application provides a method of producing a T cell having dephosphorylated proteins that regulate T cell activation through the TCR signaling pathway, wherein preferably, the proteins rely on phosphorylation as an activation mechanism for signaling in the TCR signaling pathway, preferably, the proteins are Immunoreceptor Tyrosine-based Activation Motifs (ITAMs) of a membrane-bound protein (MBP) in the TCR signaling pathway of a T cell, comprising introducing into a T cell the nucleic acid, the vector, or the composition of the present application.

In another aspect, the present application provides a method of treating a disease, wherein a therapeutically effective amount of a composition of the present application is administered to a subject in need thereof.

In another aspect, the present application provides a method of treating a disease, wherein a therapeutically effective amount of host cells of the present application is administered to a subject in need thereof.

In a further aspect, the T cell is a Jurkat cell, a primary T cell, a gamma delta T cell, or a NKT cell.

In a further aspect, the subject elicits reduced Graft-versus-Host Disease (GvHD).

In another aspect, the present application provides a method of reducing or preventing GvHD in a subject associated with the administration of one or more CAR T-cells to the subject, comprising
(1) transducing one or more of the CAR T-cells with a nucleic acid or vector or composition of the application, and
(2) administering the transduced CAR T-cells to the subject.

The technical solutions of the application have the following advantages:
1. T cell activation through the TCR signaling pathway can be regulated by the fusion proteins without degrading, knocking down, or genetically knocking out the components of TCR signaling.
2. Allogeneic CAR T cells with specific inactivation of TCR signaling, but not broad inactivation of both the TCR and CAR signaling, are obtained without degrading, knocking down, or genetically knocking out the components of the TCR signaling pathway, and GvHD in a subject associated with the administration of the CAR T-cells can be reduced or prevented.

### DESCRIPTION OF DRAWINGS

The novel features of the present disclosure are set forth with particularity in the appended claims. A better understanding of the features and advantages of the present disclosure are explained in the following detailed description in the embodiments and the examples.

FIG. 1 Cartoon schematic showing regulation of TCR signaling by the chimeric phosphatases (i.e. fusion proteins). The extracellular domains and transmembrane domains of receptor-like protein tyrosine phosphatases were exchanged for the extracellular domains and transmembrane domain of CD3ζ. Recruitment and assembly of CD3ζ into the mature TCR complex is mediated by a transmembrane domain of CD3ζ, hence resulting in TCR-specific recruitment of the phosphatase domain (i.e. intracellular phosphatase domain of the receptor-like protein tyrosine phosphatases). Once present in the TCR complex, the phosphatase domains cause dephosphorylation of CD3 ITAM domains and attenuation of TCR downstream signaling.

FIG. 2 Schematic of phosphatase chimeric constructs. We designed a version of our chimeric phosphatase constructs using the CD3ζ extracellular and transmembrane domains fused directly to intracellular phosphatase domain of either CD45 (MLB139) or CD148 (MLB140). To identify cells expressing these phosphatase constructs, we included joint expression of GFP on the construct vector separated by a P2A sequence.

FIG. 3 Example PiggyBac Construct Map. Our construct is designed with Ampicillin resistance as a selectable molecular cloning marker. Expression of the gene-of-interest is driven by the EF1α promoter sequence. A P2A sequence was used to drive joint expression of GFP as a marker for construct-transduced cells. The gene of interest and promoter sequences are flanked by “left” (5′) and “right” (3′) inverted terminal repeat (ITR) sequences which denote the boundaries of the transposable element. The ITR sequences act as a substrate for the piggyBac transposase and mediate integration into the host cell genome.

FIG. 4A-FIG. 4E CD45-based and CD148-based chimeric phosphatase expression is detectable by co-expression of GFP in Jurkat Cells. Jurkat cells transduced with MLB139, MLB140 or wildtype CD3ζ construct MLB004 were stained for CD3 expression (e.g. CD3 ε expression levels) using a fluorescently-conjugated CD3 primary antibody. CD3 staining was gated relative to an unstained wildtype Jurkat control cell population. FIG. 4A shows GFP and CD3 expression levels in a wildtype Jurkat control cell population without staining with fluorescently-conjugated CD3 primary antibody for CD3 expression (i.e. “No stain”) and without transducing the MLB139, MLB140, or wildtype CD3ζ construct MLB004. FIG. 4B shows GFP and CD3 expression levels in a wildtype Jurkat control cell population without transducing the MLB139, MLB140 or wildtype CD3ζ construct (i.e. “UTD”), but staining with fluorescently-conjugated CD3 primary antibody for CD3 expression. FIGS. 4C-4E show the comparatively high levels of GFP and CD3 expression in CD3-CD45 fusion protein (i.e. MLB139), CD3-CD148 fusion 30 protein (i.e. MLB140) and wildtype CD3ζ (i.e. MLB004) respectively.

FIG. 5A-FIG. 5B Expression of Chimeric CD45-based phosphatase (MLB139) blocks MAPK pathway activation downstream of the TCR. In FIG. 5A-FIG. 5B, there are stimulated cells (grey line marked area), unstimulated cells (filled area) and unstained cells (dotted line marked area); the stimulated cells (grey line marked area) were stimulated with 3 μL/mL ImmunoCult CD3/CD28 T cell Activator and stained for ERK1/2 (i.e. ppERK) expression by using a primary AF647-conjugated antibody; the unstimulated cells (filled area) were left unstimulated but stained with primary AF647-conjugated antibody and used as a baseline control for ERK phosphorylation; and the unstained cells (dotted line marked area), which were not stimulated with the T cell Activator and were not stained with the primary AF647-conjugated antibody, were used as a flow cytometry gating control. In FIG. 5A, the untransduced Jurkat T cells (UTD) negative for MLB139 (GFP−) showed an increase in ERK1/2 phosphorylation following TCR stimulation; FIG. 5B shows no increase in ERK1/2 phosphorylation following TCR stimulation, that is, no ERK1/2 activity (i.e. No phosphorylation of ERK1/2) in Jurkat T cells expressing MLB139 (GFP+).

FIG. 6 Expression of MLB139 in Jurkat T cells strongly inhibits T cell activation. Jurkat T cells expressing MLB139 were cultured for 6 hours or 24 hours in an OKT3 antibody-coated 96-well plate. Plates were coated with an OKT3 antibody (an activator for T cell activation) gradient with the following concentrations: 10 μg/mL, 1 μg/mL, 100 ng/mL, 10 ng/mL, 1 ng/mL, and 0 ng/mL. GFP expression was used to gate the transduced and untransduced populations. In the untransduced population (lower right), we saw an increase in CD69 upregulation with increasing OKT3 concentration. In the MLB139-transduced population (upper right), there was greatly reduced CD69 upregulation even up to the highest OKT3 antibody concentration, both at 6 hours and 24 hours.

FIG. 7A Expression of MLB140 in Jurkat T cells strongly inhibits T cell activation. Jurkat T cells expressing MLB140 were cultured for 24 hours in an OKT3-coated 96-well plate. Plates were coated with an OKT3 antibody gradient with the following concentrations: 10 μg/mL, 1 μg/mL, 100 ng/mL, 10 ng/mL, 1 ng/mL, and 0 ng/mL. GFP expression was used to gate the transduced and untransduced populations. In the untransduced population (lower right), we saw an increase in CD69 upregulation with increasing OKT3 antibody concentration. In the MLB140-transduced Jurkat population (upper right), no CD69 upregulation was observed, even at the highest OKT3 antibody concentration.

FIG. 7B shows that TCR signal inhibition is dependent on the catalytic activity of the phosphatase domain. Jurkat cells expressing the phosphatase constructs of the present application were stimulated with platebound OKT3 and CD69 upregulation was measured by flow cytometry as a readout of T cell activation. Both MLB140 and MLB139 strongly inhibited T cell activation following TCR stimulation, whereas Jurkat cells expressing MLB140.C1140S and MLB139.C840S still showed upregulation of CD69 in response to OKT3 stimulation. These results show that TCR signal inhibition is dependent on the catalytic activity of the phosphatase domains.

FIG. 8A-8B Expression of MLB139 and MLB140 detectable in primary donor-derived T cells by joint expression of GFP. Representative FACS plots of donor-derived T cells transduced to express MLB139 and MLB140. Expression of the chimeric phosphatase constructs was inferred via co-expression of GFP on the vector via a P2A sequence. Both constructs MLB139 and MLB140 had comparable levels of integration and expression in donor-derived primary T cells. FIG. 8A shows a representative histogram of MLB139-driven GFP expression in donor-derived T cells. FIG. 8B shows a representative histogram of MLB140-driven GFP expression in donor-derived T cells. In FIG. 8A-8B, the peak on the left is the negative population and the peak on the right is the positive population.

FIG. 9A-9B CD69 and CD137 expression in representative primary donor-derived T cells transduced to express MLB140. These results indicated that expression of MLB140 in primary donor-derived T cells is sufficient to attenuate T cell activation via TCR stimulation. Shown are the FACS plots for stimulated T cells from a representative donor. Donor-derived T cells were transduced via electroporation to express MLB140. Cells were then stimulated with ImmunoCult T cell activator at various concentrations: 10 μL/mL, 2.5 μL/mL, 0.6 μL/mL, or 0 uL/mL (unstimulated), which stimulates the T cells via CD3, CD28, and CD2. Cells were then harvested at 6 hours (FIG. 9A) and 24 hours (FIG. 9B) post-stimulation and stained for CD69 and CD137 (4-1BB). Gates were drawn on the cell populations to define the GFP+ and GFP− cell populations, as well as the cells expressing each of the activation markers. Expression of MLB140 was sufficient to inhibit T cell activation downstream of the TCR.

FIG. 10 MLB140 prevents primary T cell activation more efficiently than MLB139 across multiple donors. Donor-derived T cells were transduced to express MLB139 and MLB140. Cells were then stimulated with ImmunoCult T cell activator at various concentrations: 10 μL/mL, 2.5 μL/mL, 0.625 μL/mL, or 0 uL/mL (unstimulated), for 6 hours and 24 hours. Following stimulation, cells were stained for CD69 and CD137 (4-1BB) expression and analyzed by flow cytometry. Cells were gated on GFP expression to isolate the transduced and untransduced populations and additional gates were drawn on CD69 and CD137 based on the unstimulated cell populations. Here we present the fraction of positive cells in each population for CD69 and CD137 (4-1BB) in primary donor-derived T cells expressing MLB139 and MLB140, relative to the untransduced control population. We observed a strong negative regulatory effect for MLB140, but only a mild phenotype in primary cells expressing MLB139; MLB140 shows reduced fractions of both CD69 and CD137 positive cells at all activator concentrations (10 μL/mL, 2.5 μL/mL, 0.625 μL/mL) at both 6 hours and 24 hours post-stimulation; MLB139 only shows reduced fractions of CD69 at activator concentrations 0.625 μL/mL at both 6 hours and 24 hours post-stimulation, and reduced fractions of CD137 (4-1BB) at activator concentrations 10 μL/mL, 2.5 μL/mL, and 0.625 μL/mL at both 6 hours and 24 hours post-stimulation.

FIG. 11A-FIG. 11B Jurkat T cells transduced to express both MLB140 and CAR constructs. Jurkat T cells were stably co-transduced to express MLB140 and either a CEACAM5-specific CAR (LBC001) or a CLDN18.2-specific CAR (LBC010). Expression of each construct was determined by flow cytometry. MLB140 expression was determined by GFP co-expression and CAR expression was determined by staining with an anti-F(ab′)₂ antibody. FIG. 11A shows gating on GFP and F(ab′)₂ expression of doubly transduced Jurkats expressing MLB140 and LBC001. The GFP^{Lo},F(ab′)₂^{Lo} cells are the untransduced population (lower left). The GFP^{Lo} F(ab′)₂^{High} are the CAR-single positive cells (upper left). The GFP^{High},F(ab′)₂^{Lo} cells are single positive for MLB140 (lower right). The GFP^{High},F(ab′)₂^{High} cells are double positive for both MLB140 and LBC001 (CEACAM-5 CAR) (upper right). FIG. 11B shows gating on GFP and F(ab′)₂ expression of doubly transduced Jurkats expressing MLB140 and LBC010. The GFP^{Lo},F(ab′)₂^{Lo} cells are the untransduced population (lower left). The GFP^{Lo}, F(ab′)₂^{High} are the CAR-single positive cells (upper left). The GFP^{High},F(ab′)₂^{Lo} cells are single positive for MLB140 (lower right). The GFP^{High},F(ab′)₂^{High} cells are double positive for both MLB140 and LBC010 (CLDN18.2 CAR) (upper right).

FIG. 12A-FIG. 12B Co-expression of CAR construct does not impair ability of MLB140 to inhibit TCR signaling. Jurkat T cells doubly transduced with MLB140 and either LBC001 or LBC010 were cultured in 96-well plates coated with 10 μg/mL of OKT3 overnight at 37° C., 5% CO₂. Following stimulation, cells were then stained for CD69 expression and CAR expression. Gating was performed based on GFP expression and F(ab′)₂ staining and CD69 expression in each population was assessed in isolation. In FIG. 12A-FIG. 12B, untransduced cells (solid line, i.e. the topmost line) showed strong CD69 upregulation in response to OKT3 stimulation, as did the CAR-single positive cells (dot-dash line, i.e. the bottommost line). Double positive cells expressing both CAR variant and MLB140 (dashed line, i.e. the third line from top to bottom) and MLB140-single positive cells (dotted line, i.e. the second line from top to bottom) did not respond to plate-bound OKT3 stimulation, that is, co-expression of a CAR construct does not impair the ability of MLB140 to inhibit TCR signaling.

FIG. 13 Expression of MLB140 does not impair CAR-mediated Jurkat T cell activation. Jurkat T cells doubly transduced with MLB140 and LBC001 were cocultured with LoVo cells overnight at 37° C., 5% CO₂ in RPMI. Following stimulation, cells were then stained for CD3, CD69, and CAR expression. Jurkat T cells were gated on CD3 expression to exclude cocultured LoVo cells. Gating was then performed based on GFP expression and F(ab′)₂ staining and CD69 expression in each population was assessed in isolation. Untransduced Jurkat cells (solid line, i.e. the topmost line) did not upregulate CD69 in response to coculture with LoVos, nor did Jurkat cells expressing only MLB140 (dotted line, i.e. the second line from top to bottom). Jurkat cells expressing only the CEACAM5-specific CAR (dot-dash line, i.e. the bottommost line) or expressing both the CAR and MLB140 (dashed line, i.e. the third line from top to bottom) both upregulated CD69 expression in response to coculture with LoVo cells.

FIG. 14 Schematic representation of puromycin-resistant PhosphoTAC constructs based on CD3ζ, CD3ε, CD3δ, and CD3γ (MLB140.1, MLB178, MLB180, and MLB181)

We designed CD148 phosphatase fusion proteins (MLB140.1, MLB178, MLB180, and MLB181) based on each of the CD3 isotypes. Each of these constructs utilized the extracellular and transmembrane domains of CD3 fused to the intracellular domain of CD148. This sequence was separated from a puromycin selection marker (“puro.”) by a P2A sequence. At the 3′ end of the cassette, we also included a GFP fluorescent marker separated from the puromycin resistance marker by an E2A sequence.

FIG. 15 CD3ζ, CD3ε, CD3δ, and CD3γ fusion proteins with CD148 intracellular domain (MLB140.1, MLB178, MLB180, and MLB181) inhibit T cell activation

Representative flow cytometry plots from OKT3 activation experiments are shown. Jurkat T cells transduced with MLB140.1, MLB178, MLB180 and MLB181 were stimulated with varying concentrations of OKT3. Shown in FIG. 15 are the response profiles to 10 μg/mL of plate bound OKT3 or the negative control cells (i.e., 0 μg/mL of OKT3). GFP expression in FIG. 15 was used to gate the transduced and untransduced populations. In the untransduced population, we saw an increase in CD69 upregulation with increasing OKT3 concentration (lower right of each panel). In the transduced population, there was greatly reduced CD69 upregulation even up to the higher OKT3 antibody concentrations (upper right of each panel). In summary, FIG. 15 shows all of the constructs tested in this assay (MLB140.1, MLB178, MLB180, and MLB181) were able to inhibit T cell activation as was observed in the decreased fraction of CD69 positive cells in the GFP positive population.

FIG. 16 CD3ζ, CD3ε, CD3δ, and CD3γ fusion proteins with CD148 intracellular domain (MLB140.1, MLB178, MLB180, and MLB181) are equally efficient at preventing T cell activation

Geometric MFI of CD69 expression (gMFI CD69 (a.u.)) was quantified following overnight OKT3 stimulation (10 μg/mL, 1 μg/mL, 0.1 μg/mL, or 0 μg/mL of OKT3 antibody) to determine the degree of T cell activation. CD69 expression was quantified in both the GFP positive and GFP negative fractions for populations of Jurkat cells expressing constructs MLB140.1, MLB178, MLB180, and MLB181. It can be seen from FIG. 16 that all of the constructs MLB140.1, MLB178, MLB180, and MLB181 were equally efficient at inhibiting T cell activation.

FIG. 17 Schematic design of AC7 construct (contains both MLB140′ phosphatase fusion protein and CEACAM-5 CAR)

Construct MLB140′ (that is, SP-CD3ζ ex/tm-CD148 int-P2A, or MLB140 with no GFP) was expressed at the 5′ end of the joint expression vector while the CEACAM-5 specific CAR (that is, CD8α signal peptide -MN14op scFv-CD8α hinge-CD8αTM-4-1BB-CD3ζ int) was expressed at the 3′ end of the vector. The two components were separated by a P2A sequence. We refer to this construct as AC7 construct (contains both MLB140′ and CEACAM-5 CAR).

FIG. 18A-FIG. 18C FACS expression profiles of CD3ζ-CD148 phosphatase fusion protein and CEACAM-5 CAR

Primary T cells derived from healthy donors were transduced via electroporation to express 1) the parental control CAR: MLB010 CAR (that is, CEACAM-5 CAR/GFP, or CD8α signal peptide -MN14op scFv-CD8α hinge-CD8αTM-4-1BB-CD3ζ int-P2A-GFP), or 2) AC7 (MLB140′-CEACAM-5 CAR, or SP-CD3ζ ex/tm-CD148 int-P2A-SP-CEACAM-5 scFv-CD8α hinge-CD8αTM-4-1BB-CD3 int) and expression was evaluated at day 9 post-transduction by FACS labeling using a fluorescently-conjugated anti-F(ab′)₂ antibody. Additionally, the parental CAR MLB010 also had a GFP transduction marker (i.e. CEACAM-5 CAR/GFP). FIG. 18A shows GFP and F(ab′)₂ expression levels in a Primary T cell population without transduction of MLB010 CAR or AC7 (i.e. UTD); FIG. 18B shows gating on GFP and F(ab′)₂ expression of MLB010 CAR-transduced Primary T cells. FIG. 18C shows gating on GFP and F(ab′)₂ expression of AC7-transduced primary T cells.

FIG. 19 AC7 CAR construct shows antigen-specific cytotoxicity against LoVo target cells

CAR T cells derived from healthy donors and expressing AC7 or MLB010 were incubated for 24 hours with LoVo cells at an E:T ratio of 3:1, 1:1, or 0.3:1. LoVo target cells express luciferase and cytotoxicity was measured as a decrease in bioluminescence in the CAR-T cell treatment group relative to the untreated control cells. We observed that there was antigen-specific killing by CAR T cells expressing construct AC7, but that the killing efficiency was lower than T cells expressing the parental CEACAM-5 specific CAR, MLB010.

FIG. 20 AC7 CAR T cells show no TNFα secretion following incubation with target cells

CAR T cells derived from healthy donors and expressing AC7 or MLB010 were incubated for 24 hours with LoVo cells at an E:T ratio of 3:1, 1:1, or 0.3:1. Supernatant was harvested and assayed for TNFα by ELISA.

FIG. 21 AC7 CAR T cells show no IFNγ secretion following incubation with target cells

CAR T cells derived from healthy donors and expressing AC7 or MLB010 were incubated for 24 hours with LoVo cells at an E:T ratio of 3:1, 1:1, or 0.3:1. Supernatant was harvested and assayed for IFNγ by ELISA.

FIG. 22A Phospho-flow cytometry for both MLB139.1 and MLB140.1 for CD3zeta (CD3 ζ) phosphorylation. The stimulated Jurkat T cells (the upper left and upper right histograms) showed decreased CD3zeta phosphorylation in the transduced populations (solid gray line marked area) compared to the untransduced populations (dashed black line marked area), whereas unstimulated Jurkat T cells (the lower left and lower right histograms) showed no difference in CD3zeta phosphorylation between the transduced populations and the untransduced populations. In all of the histogram plots, the shaded gray histogram is the secondary-only control population.

FIG. 22B shows CD3ζ dephosphorylation is dependent of catalytic activity of chimeric phosphatases. Cells were gated on GFP expression to identify cells expressing MLB140.C1140S (solid gray line) or MLB139.C840S (solid gray line), and untransduced control cells (dashed black line). Both MLB140.C1140S or MLB139.C840S showed no difference in CD3zeta phosphorylation between the transduced populations and the untransduced populations. In all of the histogram plots, the shaded gray histogram is the secondary-only control population.

FIG. 23A shows Phospho-Western Blot for SLP76. FIG. 23B shows pSLP-76 fluorescence intensity was quantified relative to the band intensity of the β-actin loading control.

The stimulated Jurkat T cells (+) showed decreased pSLP-76 (i.e. SLP76) expression in the populations transduced with MLB140.1 than the untransduced wild type (WT), whereas unstimulated Jurkat T cells (−) showed no difference in pSLP-76 expression between the transduced populations and the untransduced populations.

### GENERAL DEFINITIONS

Unless defined otherwise, technical and scientific terms used herein have the same meaning as generally used in the art to which this application belongs. For purposes of interpreting this specification, the following definitions will apply and whenever appropriate, terms used in the singular will also include the plural and vice versa. As used herein and in the appended claims, the singular forms “a”, “an”, and “the” also refer to the plural forms unless the context clearly dictates otherwise, e.g., reference to “a host cell” includes a plurality of such host cells.

As used herein, a “T cell receptor (TCR) complex”, otherwise known as the TCR/CD3 complex, is a multimeric complex on the T-cell surface whose activation leads to the activation of the T-cell. The complex comprises (i) TCR, and (ii) CD3 T-cell co-receptor. As set forth below, the TCR comprises alpha (α) and beta (β) chains. The CD3 T-cell co-receptor comprises a CD3-gamma (CD3γ) chain, a CD3-delta (CD3δ) chain, two CD3-epsilon (CD3ε) chains and two CD3-zeta (CD3ζ) chains as accessory molecules.

TCRs allow for the antigen-specific activation of T-cells. Every T-cell expresses clonal TCRs which recognize a specific peptide/MHC complex during physical contact between a T cell and an antigen-presenting cell (APC) (via MHC class II) or any other cell type (via MHC class I). TCR triggering occurs when the TCR antigen receptor complex encounters cognate peptide-bound Major Histocompatibility Complex (pMHC). The TCR complex is comprised of the TCRα and TCRβ antigen recognition domains and CD3ζ, σ, γ, and ε signaling domains. Signal transmission through the TCR is mediated by phosphorylation and dephosphorylation of Immunoreceptor Tyrosine-based Activation Motifs (ITAMs). The TCR is a disulfide-linked membrane-anchored heterodimeric protein normally consisting of the highly variable alpha (α) and beta (β) chains. Each chain of the TCR comprises two extracellular domains: a variable (V) region and a constant (C) region, both of immunoglobulin superfamily (IgSF) domain forming antiparallel beta-sheets. The constant region is proximal to the cell membrane, followed by a transmembrane region and a short cytoplasmic tail, while the variable region binds to the peptide/MHC complex. The variable domain of the TCR alpha-chain and the TCR beta-chain each have three hypervariable or complementarity determining regions (CDRs) that contribute to the TCR's specificity for a particular peptide/MHC complex. The variable region of the beta-chain also has an additional area of hypervariability (HV4) that does not normally contact antigen.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. In addition, antibody fragments comprise single chain polypeptides having the characteristics of a VH domain, namely being able to assemble together with a VL domain, or of a VL domain, namely being able to assemble together with a VH domain into a functional antigen binding site and thereby provide the antigen binding property of full-length antibodies.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the application are used to delay development of a disease or to slow the progression of a disease.

The term “cancer” or “tumor” as used herein refers to proliferative diseases, such as ovarian cancer, pancreatic cancer, colon cancer, colorectal cancer, lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to limit the scope of the present application.

The present application provides novel fusion proteins with particularly advantageous properties such as dephosphorylating proteins that regulate T cell activation through the TCR signaling pathway when expressed in a host cell.

In the present application, the dephosphorylation of proteins that regulate T cell activation through the TCR signaling pathway (wherein preferably, the proteins rely on phosphorylation as an activation mechanism for signaling in the TCR signaling pathway, such as one or more subunits or structural domains of membrane-bound protein (MBP) of the T cell, kinases and/or scaffold proteins) is achieved by a dephosphorylation protein, wherein the dephosphorylation protein comprises one or more subunits or structural domains of receptor-like protein tyrosine phosphatases (e.g. CD45 or CD148) or variants thereof. The targeting specificity of the dephosphorylation protein for the proteins that regulate T cell activation through the TCR signaling pathway is mediated by a linking protein (e.g. transmembrane domain of one or more subunits of a membrane-bound protein (MBP)). T cell activation through the TCR signaling pathway thus can be regulated by the fusion proteins without degrading, knocking down, or genetically knocking out the components of TCR signaling. Allogeneic CAR T cells which specifically inactivate the TCR signaling, but do not broadly inactivate both the TCR and CARs signaling, can be obtained without degrading, knocking down, or genetically knocking out the components of TCR signaling, and GvHD in a subject associated with the administration of the CAR T-cells can be reduced or prevented.

This is a novel TCR signaling regulation technology that is based on protein dephosphorylation specific to components of TCR signaling but not broadly to components of CARs signaling, and does not involve degrading, knocking down, or genetically knocking out the components of TCR signaling.

### (1) Fusion Proteins of the Present Application

The present application discloses a fusion protein comprising:
\textcircled{1} the transmembrane domain of one or more of CD3ζ, CD3γ, CD3δ, CD3ε, TCRα or TCRβ or variants thereof; and \textcircled{2} the intracellular phosphatase domain of CD45 or CD148 or a variant thereof; or
\textcircled{2} the extracellular domain of one or more of CD3ζ, CD3γ, CD3δ, CD3ε, TCRα or TCRβ or variants thereof; the transmembrane domain of one or more of CD3ζ, CD3γ, CD3δ, CD3ε, TCRα or TCRβ or variants thereof; and \textcircled{2} the intracellular phosphatase domain of CD45 or CD148 or a variant thereof. \textcircled{1} the extracellular domain of one or more of CD3ζ, CD3γ, CD3δ, CD3ε, TCRα or TCRβ or variants thereof; the transmembrane domain of one or more of CD3ζ, CD3γ, CD3δ, CD3ε, TCRα or TCRβ or variants thereof; \textcircled{2} the intracellular phosphatase domain of CD45 or CD148 or a variant thereof; and \textcircled{3} a puromycin selection marker and/or GFP.

In some embodiments, the fusion protein further comprises a signal peptide sequence at the N-terminal. In some embodiments, the signal peptide sequence is located at the N-terminal of the extracellular domain.

In a non-limiting example, the signal peptide sequence comprises an amino acid sequence at least about 95%, 96%, 97%, 98%, 99% or 100% identical to the amino acid sequence shown in any one of SEQ ID NOs. 1, 26, 29 and 32;
preferably the CD3ζ signal peptide sequence comprises an amino acid sequence at least about 95%, 96%, 97%, 98%, 99% or 100% identical to the amino acid sequence shown in SEQ ID NO. 1, the CD3ε signal peptide sequence comprises an amino acid sequence at least about 95%, 96%, 97%, 98%, 99% or 100% identical to the amino acid sequence shown in SEQ ID NO. 26, the CD3γ signal peptide sequence comprises an amino acid sequence at least about 95%, 96%, 97%, 98%, 99% or 100% identical to the amino acid sequence shown in SEQ ID NO. 29, and the CD3δ signal peptide sequence comprises an amino acid sequence at least about 95%, 96%, 97%, 98%, 99% or 100% identical to the amino acid sequence shown in SEQ ID NO. 32.

In some embodiments, the extracellular domain of CD3ζ comprises an amino acid sequence at least about 95%, 96%, 97%, 98%, 99% or 100% identical to the amino acid sequence shown in SEQ ID NO. 2, the extracellular domain of CD3ε comprises an amino acid sequence at least about 95%, 96%, 97%, 98%, 99% or 100% identical to the amino acid sequence shown in SEQ ID NO. 27, the extracellular domain of CD3γ comprises an amino acid sequence at least about 95%, 96%, 97%, 98%, 99% or 100% identical to the amino acid sequence shown in SEQ ID NO. 30, and the extracellular domain of CD3δ comprises an amino acid sequence at least about 95%, 96%, 97%, 98%, 99% or 100% identical to the amino acid sequence shown in SEQ ID NO. 33.

In some embodiments, the transmembrane domain of CD3ζ comprises an amino acid sequence at least about 95%, 96%, 97%, 98%, 99% or 100% identical to the amino acid sequence shown in SEQ ID NO. 3, the transmembrane domain of CD3ε comprises an amino acid sequence at least about 95%, 96%, 97%, 98%, 99% or 100% identical to the amino acid sequence shown in SEQ ID NO. 28, the transmembrane domain of CD3γ comprises an amino acid sequence at least about 95%, 96%, 97%, 98%, 99% or 100% identical to the amino acid sequence shown in SEQ ID NO. 31, and the transmembrane domain of CD3δ comprises an amino acid sequence at least about 95%, 96%, 97%, 98%, 99% or 100% identical to the amino acid sequence shown in SEQ ID NO. 34.

In some embodiments, the intracellular phosphatase domain of CD45 comprises an amino acid sequence at least about 95%, 96%, 97%, 98%, 99% or 100% identical to the amino acid sequence shown in SEQ ID NO. 4.

In some embodiments, the intracellular phosphatase domain of CD148 comprises an amino acid sequence at least about 95%, 96%, 97%, 98%, 99% or 100% identical to the amino acid sequence shown in SEQ ID NO. 5.

In some embodiments, the puromycin selection marker comprises an amino acid sequence at least about 95%, 96%, 97%, 98%, 99% or 100% identical to the amino acid sequence shown in SEQ ID NO. 25.

a. Linking Protein: The Transmembrane Domain of One or More of CD3ζ, CD3γ, CD3δ, CD3δ, TCRα or TCRβ or Variants Thereof, or an Extracellular Domain and a Transmembrane Domain of One or More of CD3ζ, CD3γ, CD3δ, CD3δ, TCRα or TCRβ or Variants Thereof

In some embodiments, the transmembrane domain of CD3ζ comprises an amino acid sequence at least about 95%, 96%, 97%, 98%, 99% or 100% identical to the amino acid sequence shown in SEQ ID NO. 3, the transmembrane domain of CD3ε comprises an amino acid sequence at least about 95%, 96%, 97%, 98%, 99% or 100% identical to the amino acid sequence shown in SEQ ID NO. 28, the transmembrane domain of CD3γ comprises an amino acid sequence at least about 95%, 96%, 97%, 98%, 99% or 100% identical to the amino acid sequence shown in SEQ ID NO. 31, and the transmembrane domain of CD3δ comprises an amino acid sequence at least about 95%, 96%, 97%, 98%, 99% or 100% identical to the amino acid sequence shown in SEQ ID NO. 34.

In some embodiments, extracellular domain of CD3ζ comprises an amino acid sequence at least about 95%, 96%, 97%, 98%, 99% or 100% identical to the amino acid sequence shown in SEQ ID NO. 2, the extracellular domain of CD3ε comprises an amino acid sequence at least about 95%, 96%, 97%, 98%, 99% or 100% identical to the amino acid sequence shown in SEQ ID NO. 27, the extracellular domain of CD3γ comprises an amino acid sequence at least about 95%, 96%, 97%, 98%, 99% or 100% identical to the amino acid sequence shown in SEQ ID NO. 30, and the extracellular domain of CD3δ comprises an amino acid sequence at least about 95%, 96%, 97%, 98%, 99% or 100% identical to the amino acid sequence shown in SEQ ID NO. 33.

When the transmembrane domain of CD3ζ or the extracellular domain and a transmembrane domain of CD3ζ is used as the linking protein of the fusion protein, the fusion protein may not only be specific to CD3ζ dephosphorylation, but also have dephosphorylation effect on other components of TCR signaling (for example, CD3γ, CD3δ, CD3δ, and kinase, such as ZAP70, and scaffold proteins, such as LAT and/or SLP76) without inactivating CAR signaling. Moreover, the same effect can be achieved when one of the transmembrane domains or the extracellular domain and a transmembrane domain of CD3δ, CD3γ, CD3δ, TCRα or TCRβ is used as the linking protein of the fusion protein.

b. Dephosphorylation Protein: One or More Subunits or Structural Domains of Receptor-Like Protein Tyrosine Phosphatases (e.g. CD45 or CD148) or Variants Thereof.

In some embodiments, the intracellular phosphatase domain of CD45 comprises an amino acid sequence at least about 95%, 96%, 97%, 98%, 99% or 100% identical to the amino acid sequence shown in SEQ ID NO. 4.

In some embodiments, the intracellular phosphatase domain of CD148 comprises an amino acid sequence at least about 95%, 96%, 97%, 98%, 99% or 100% identical to the amino acid sequence shown in SEQ ID NO. 5.

c. Transmembrane Domain

“Transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans.

The function of a transmembrane domain in the fusion protein is to anchor said fusion protein to the cell surface and mediate recruitment of the fusion protein to the TCR complex, and thus any transmembrane domain fulfilling that function can be used in the present application.

A transmembrane domain suitable for the present application can be selected from the following: the transmembrane domain of one or more of CD3ζ, CD3γ, CD3δ, CD3δ, TCRα or TCRβ or variants thereof.

Optionally, the transmembrane domain of CD3ζ comprises an amino acid sequence at least about 95%, 96%, 97%, 98%, 99% or 100% identical to the amino acid sequence shown in SEQ ID NO. 3, the transmembrane domain of CD3ε comprises an amino acid sequence at least about 95%, 96%, 97%, 98%, 99% or 100% identical to the amino acid sequence shown in SEQ ID NO. 28, the transmembrane domain of CD3γ comprises an amino acid sequence at least about 95%, 96%, 97%, 98%, 99% or 100% identical to the amino acid sequence shown in SEQ ID NO. 31, and the 30 transmembrane domain of CD3δ comprises an amino acid sequence at least about 95%, 96%, 97%, 98%, 99% or 100% identical to the amino acid sequence shown in SEQ ID NO. 34.

(2) Nucleic Acid Encoding Fusion Proteins, Vector Comprising Said Nucleic Acid, and Composition Comprising said Nucleic Acid or Vector

The present application provides a nucleic acid encoding the fusion proteins described herein. The nucleic acid encoding the fusion proteins can be easily prepared from an amino acid sequence of the specified fusion proteins by a conventional method. A nucleotide sequence encoding an amino acid sequence can be obtained from the aforementioned NCBI RefSeq IDs or accession numbers of GenBank for an amino acid sequence of each domain, and the nucleic acid of the present disclosure can be prepared using a standard molecular biological and/or chemical procedure. For example, based on the nucleotide sequence, a nucleic acid can be synthesized, and the nucleic acid of the present disclosure can be prepared by combining DNA fragments which are obtained from a cDNA library using a polymerase chain reaction (PCR).

The nucleic acid of the present disclosure can be linked to another nucleic acid so as to be expressed under control of a suitable promoter. Examples of the promoter include a promoter that constitutively promotes the expression of a gene or operatively linked construct, or a promoter that induces the expression of a gene or operatively linked construct by the action of a drug or the like (e.g. tetracycline, ampicillin or doxorubicin). In order to attain efficient transcription of the nucleic acid, the nucleic acid of the present disclosure can also be linked to other regulatory elements that cooperate with a promoter or a transcription initiation site, for example, a nucleic acid comprising an enhancer sequence or a terminator sequence. In addition to the nucleic acid of the present disclosure, a gene that can be a marker for confirming expression of the nucleic acid (e.g. a drug resistance gene, a gene encoding a reporter enzyme, or a gene encoding a fluorescent protein) may be incorporated.

Furthermore, in addition to the nucleic acid encoding the fusion protein, a gene encoding a purification selection marker can also be incorporated. In one embodiment, a fusion protein expression cassette is provided, which comprises a nucleic acid encoding both the fusion protein of the present application and a purification selection marker; more preferably, the purification selection marker is a puromycin selection marker, more preferably, the puromycin selection marker comprises an amino acid sequence at least about 95%, 96%, 97%, 98%, 99% or 100% identical to the amino acid sequence shown in SEQ ID NO. 25.

In an embodiment, the nucleic acid is codon-optimized nucleic acid for expression in a particular host.

The present application further provides a vector comprising nucleic acid encoding the fusion protein or the fusion protein expression cassette described herein. The term “vector”, “expression vector”, and “expression construct” or “construct” are used interchangeably, and are both defined to be a plasmid, virus, or other nucleic acid designed for protein expression in a cell. The vector or construct is used to introduce a gene into a host cell whereby the vector will interact with polymerases in the cell to express the protein encoded in the vector/construct. The expression vector and/or expression construct may exist in a cell extrachromosomally or integrated into the chromosome. When integrated into the chromosome, the nucleic acids comprising the expression vector or expression construct will be an expression vector or expression construct.

The present application further provides a composition comprising at least one nucleic acid or at least one vector described herein.

The present application further provides a composition comprising a first nucleic acid and a second nucleic acid, or a composition comprising a first vector having a first nucleic acid and a second vector having a second nucleic acid, or a composition comprising a vector having a first nucleic acid and a second nucleic acid, wherein
(1) the first nucleic acid encodes the fusion protein of the application, and
(2) the second nucleic acid encodes a chimeric antigen receptor (CAR) comprising:
(a) an extracellular ligand-binding domain comprising a single chain variable fragment (scFv) specifically binding to a predetermined antigen;
(b) a transmembrane domain, preferably CD8α, CD28, 4-1BB or IL2Rβ transmembrane domain, more preferably CD8α transmembrane domain; and
(c) a cytoplasmic segment comprising one or more signaling domains, preferably comprising a 4-1BB signaling domain and a CD3ζ signaling domain.

In some embodiments, the predetermined antigen is a tumor-related antigen.

In some embodiments, the tumor-related antigen is selected from the following group: CEA, Claudin 18.2, GPC3, Receptor tyrosine kinase-like Orphan Receptor 1 (ROR1), CD38, CD19, CD20, CD22, BCMA, CAIX, CD446, CD133, EGFR, EGFRvIII, EpCam, GD2, EphA2, Her1, Her2, ICAM-1, IL13Ra2, Mesothelin, MUC1, MUC16, NKG2D, PSCA, NY-ESO-1, MART-1, WT1, MAGE-A10, MAGE-A3, MAGE-A4, EBV, NKG2D, PD1, PD-L1, CD25, IL-2, and CD3.

In some embodiments, the tumor-related antigen is CEA, and optionally CEACAM5.

In some embodiments, the single chain variable fragment (scFv) comprises an amino acid sequence at least about 95%, 96%, 97%, 98%, 99% or 100% identical to the amino acid sequence shown in SEQ ID NO. 12 or 13.

In some embodiments, the CD8α transmembrane domain comprises an amino acid sequence at least about 95%, 96%, 97%, 98%, 99% or 100% identical to the amino acid sequence shown in SEQ ID NO. 15.

In some embodiments, the 4-1BB signaling domain comprises an amino acid sequence at least about 95%, 96%, 97%, 98%, 99% or 100% identical to the amino acid sequence shown in SEQ ID NO. 16.

In some embodiments, the CD3ζ signaling domain comprises an amino acid sequence at least about 95%, 96%, 97%, 98%, 99% or 100% identical to the amino acid sequence shown in SEQ ID NO. 17.

In some embodiments, LBC001 (MN14op scFv-CD8α hinge-CD8αTM-4-1BB-CD3ζ int) comprises an amino acid sequence at least about 95%, 96%, 97%, 98%, 99% or 100% identical to the amino acid sequence shown in SEQ ID NO. 18.

In some embodiments, LBC010 (841 scFv-CD8α hinge-CD8αTM-4-1BB-CD3ζ int) comprises an amino acid sequence at least about 95%, 96%, 97%, 98%, 99% or 100% identical to the amino acid sequence shown in SEQ ID NO. 19.

### (3) Host Cells

The present application provides a host cell comprising the nucleic acid or vector or composition described herein. Thus, when expressed, the fusion proteins described herein will be expressed on the surface of the host cells for cell therapy.

In some embodiments, the host cell is an allogeneic cell.

In some embodiments, the host cell is a mammalian cell, preferably a primate cell, more preferably a human cell.

In some embodiments, the host cell is a T cell; preferably the T cell is selected from a Jurkat cell, a primary T cell, a gamma delta T cell, or a NKT cell.

### (4) Pharmaceutical Composition

Pharmaceutical compositions of the present application comprise a fusion protein-expressing cell, preferably a fusion protein and a CAR-expressing cell, as described herein, in combination with one or more pharmaceutically or physiologically acceptable carriers, diluents or excipients. Such compositions may comprise buffers such as neutral-buffered saline, phosphate-buffered saline and the like; carbohydrates such as glucose, mannose, sucrose, dextrans, or mannitol; proteins, polypeptides or amino acids such as glycine; antioxidants; chelating agents such as EDTA or glutathione; adjuvants (e.g., aluminum hydroxide); and preservatives. Compositions of the present application are in one aspect formulated for intravenous administration.

Pharmaceutical compositions of the present application may be administered in a manner appropriate to the disease to be treated (or prevented). The quantity and frequency of administration will be determined by factors such as the condition of the patient, and the type and severity of the patient's disease, although appropriate dosages may be determined through clinical trials.

Suitable pharmaceutically acceptable excipients are well known to a person skilled in the art. Examples of pharmaceutically acceptable excipients include phosphate-buffered saline (e.g. 0.01 M phosphate, 0.138 M NaCl, 0.0027 M KCl, pH 7.4), an aqueous solution containing a mineral acid salt such as a hydrochloride, a hydrobromide, a phosphate, or a sulfate, saline, a solution of glycol or ethanol, and a salt of an organic acid such as an acetate, a propionate, a malonate or a benzoate. In some embodiments, an adjuvant such as a wetting agent or an emulsifier, and a pH buffering agent can also be used. In some embodiments, the pharmaceutically acceptable excipients described in Remington's Pharmaceutical Sciences can be appropriately used. The composition of the present application can be formulated into a known form suitable for parenteral administration, for example, injection or infusion. In some embodiments, the composition of the present application may comprise formulation additives such as a suspending agent, a preservative, a stabilizer and/or a dispersant, and a preservation agent for extending a shelf-life term during storage.

### (5) Treatment Method

The present application provides a method of treating disease in a subject in need thereof, comprising administering to the subject an effective amount of the pharmaceutical composition described herein.

In some embodiments, the disease is cancer.

In some embodiments, the cancer is a hematological malignancy or a solid tumor.

In some embodiments, the cancer is ovarian cancer, pancreatic cancer, colon cancer, colorectal cancer, lymphoma, esophageal cancer, lung cancer, ovarian cancer, hepatic cancer, head-neck cancer, or cancer of the gallbladder.

### (6) Method of Producing a Cell Having Dephosphorylated Subunits or Structural Domains of a Membrane-Bound Protein (MBP)

The present application provides a method of producing a cell having dephosphorylated subunits or structural domains of a membrane-bound protein (MBP), comprising introducing into a cell a nucleic acid, a vector, or a composition described herein.

In some embodiments, the host cell is a mammalian T cell, preferably a human T cell.

In some embodiments, the host cell is a Jurkat cell, a primary T cell, a gamma delta T cell, or a NK T cell.

In some embodiments, the host cell is an allogeneic T cell.

In some embodiments, the allogeneic CAR T cell has dephosphorylated subunits or structural domains of a membrane-bound protein (MBP) (e.g. dephosphorylated Immunoreceptor Tyrosine-based Activation Motifs (ITAMs) of CD3 in the TCR/CD3 complex), and a CAR as mentioned in (2) above.

### (7) Method of Reducing or Preventing GvHD

The present application provides a method of reducing or preventing GvHD in a subject associated with the administration of one or more CAR T-cells to the subject, comprising:
\textcircled{1} transducing the one or more CAR T-cells with a nucleic acid or a vector described herein, and
\textcircled{2} administering the transduced CAR T-cells to the subject.
   (8) Method of Dephosphorylating Proteins that Regulate T Cell Activation Through the TCR Signaling Pathway

The present application provides a method of dephosphorylating proteins that regulate T cell activation through the TCR signaling pathway, wherein preferably, the proteins rely on phosphorylation as an activation mechanism for signaling in the TCR signaling pathway, preferably, the proteins are Immunoreceptor Tyrosine-based Activation Motifs (ITAMs) of a membrane-bound protein (MBP) of a cell, comprising introducing into a cell the nucleic acid, the vector, or the composition of the present application.

### Example 1

A-1. Design of Chimeric Phosphatase Constructs (i.e. Fusion Protein Constructs)

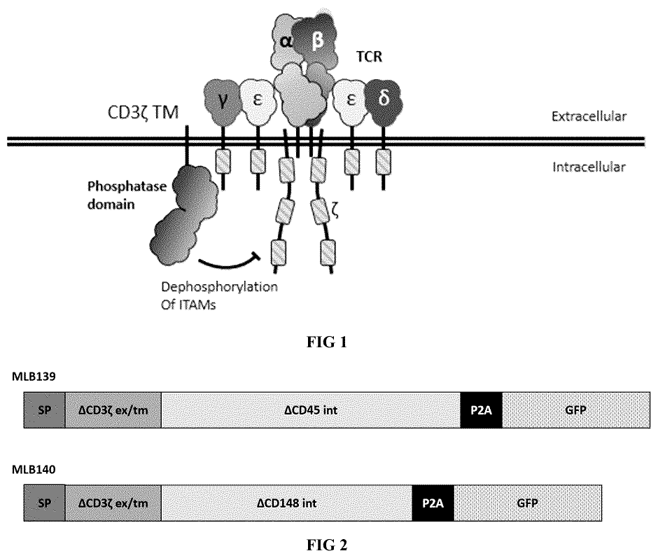

We designed a list of constructs to eliminate TCR signaling through the targeted recruitment of phosphatases to the TCR complex through modification of transmembrane domains and extracellular domains of the phosphatases (i.e. receptor-like protein tyrosine phosphatases) (FIG. 1). CD45 and CD148 are phosphatases that are naturally expressed in lymphocytes and regulate their responsiveness to antigenic stimulation. The activity of both CD45 and CD148 is biophysically regulated by their large extracellular domains. We generated several chimeric fusion protein constructs with the extracellular and transmembrane domains of CD3 at the N-terminus fused to a C-terminal phosphatase domain (FIG. 2). MLB139 and MLB140 are derived from CD45 and CD148, respectively (sequences of them are shown in Table 1).
1. MLB139, Extracellular and transmembrane domains of CD3 at the N-terminus fused to CD45 phosphatase intracellular domains at the C-terminal (SP-CD3ζ ex/tm-CD45 int-P2A-GFP);
2. MLB140, Extracellular and transmembrane domains of CD3 at the N-terminus fused to CD148 phosphatase intracellular domains at the C-terminal (SP-CD3ζ ex/tm-CD148 int-P2A-GFP).
   A-2. Design of CD3ε-CD148, CD3γ-CD148, and CD3δ-CD148 Chimeric Phosphatase Constructs (i.e. Fusion Protein Constructs)

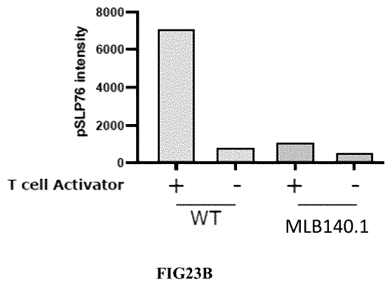

We wanted to further examine the effect of phosphatase recruitment to the TCR to determine whether or not our observations were dependent on phosphatase activity, as opposed to merely dependent on the substitution of the endogenous CD3 chain with our chimeric protein. We designed an additional 3 constructs to test this hypothesis. These constructs were designed using the CD3δ, CD3ε, and CD3γ extracellular domains and transmembrane domains directly fused to the N-terminus of the CD148 intracellular domain (FIG. 14). Additionally, these constructs were designed with a Puromycin resistance marker (i.e. Puro.) for puromycin selection and enrichment. We also designed a puromycin resistant version of the CD3ζ-CD148 construct (MLB140.1) (FIG. 14). Sequence information is provided in Table 1. The following specific designs were synthesized and tested:
1. MLB178, Extracellular and transmembrane domains of CD3ε at the N-terminus fused to CD148 phosphatase intracellular domains at the C-terminus (SP-CD3ε ex/tm-CD148 int-P2A-Puro-E2A-GFP);
2. MLB180, Extracellular and transmembrane domains of CD3δ at the N-terminus fused to CD148 phosphatase intracellular domains at the C-terminus (SP-CD38 ex/tm-CD148 int-P2A-Puro-E2A-GFP);
3. MLB181, Extracellular and transmembrane domains of CD3γ at the N-terminus fused to CD148 phosphatase intracellular domains at the C-terminus (SP-CD3γ ex/tm-CD148 int-P2A-Puro-E2A-GFP);
4. MLB140.1 (a puromycin resistance version of the MLB140), Extracellular and transmembrane domains of CD3 at the N-terminus fused to CD148 phosphatase intracellular domains at the C-terminus (SP-CD3ζ ex/tm-CD148 int-P2A-Puro-E2A-GFP).

### B. Design and Generation of Reference Constructs

1. MLB004, SP-wildtype CD3ζ (i.e. CD3ζ ex/tm/int)-P2A-GFP

We designed a reference construct by linking extracellular and transmembrane domains of CD3 with the intracellular domain of CD3ζ.

### C. Design and Generation of CAR

1. LBC001 (MN14op scFv-CD8α hinge-CD8αTM-4-1BB-CD3ζint)
2. LBC010 (841 scFv-CD8α hinge-CD8αTM-4-1BB-CD3ζ int)
3. MLB010 (i.e. CEACAM-5 CAR/GFP or CD8α signal peptide -MN14op scFv-CD8α hinge-CD8αTM-4-1BB-CD3ζ int-P2A-GFP)

The sequences of components of LBC001, LBC010, and MLB010 are shown in Table 1.

### D. Design and Generation of AC7 Construct

AC7. SP-CD3ζ ex/tm-CD148 int-P2A-SP-CEACAM-5 scFv-CD8α hinge-CD8αTM-4-1BB-CDKint

### Example 2

Abrogation of Upstream TCR Signaling in Jurkat T Cells Transduced with Chimeric Phosphatases

### 1. Generation of the Chimeric CD3ζ-CD45 or CD3ζ-CD148 Fusion Protein

The peptide sequences of CD3ζ (CD247), CD45, and CD148 were analyzed and the signal peptide, extracellular, transmembrane, and intracellular sequences were identified based on the reported peptide sequences. The chimeric phosphatases were designed as direct fusions of the CD45 or CD148 intracellular domain sequences to the signal peptide, extracellular and 15 transmembrane sequences of CD3ζ. DNA was synthesized based on the chimeric phosphatase DNA sequences with codons optimized for human expression. The synthesized DNA fragment was cloned into the PiggyBac expression vector upstream of the P2A GFP sequence.

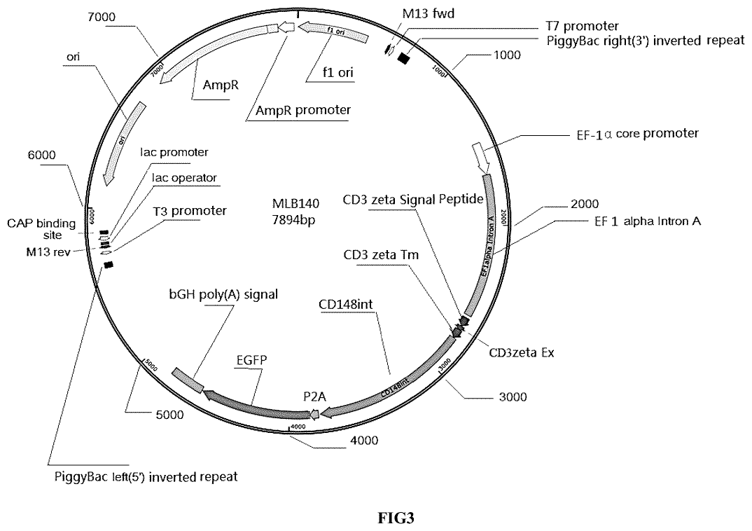

Jurkat T cells were electroporated with the PiggyBac transposon, the construct bearing MLB139, MLB140, or wildtype CD3ζ (MLB004) and Hyperactive PiggyBac transposase mRNA, then cultured at 37° C. and 5% CO₂. Expression of the chimeric phosphatases was inferred by co-expression of GFP and through TCR stimulation functional assays. PiggyBac construct bearing MLB140 is shown in FIG. 3.

### 2. Expression Levels of Chimeric CD3-CD45 or CD3-CD148 Fusion Protein

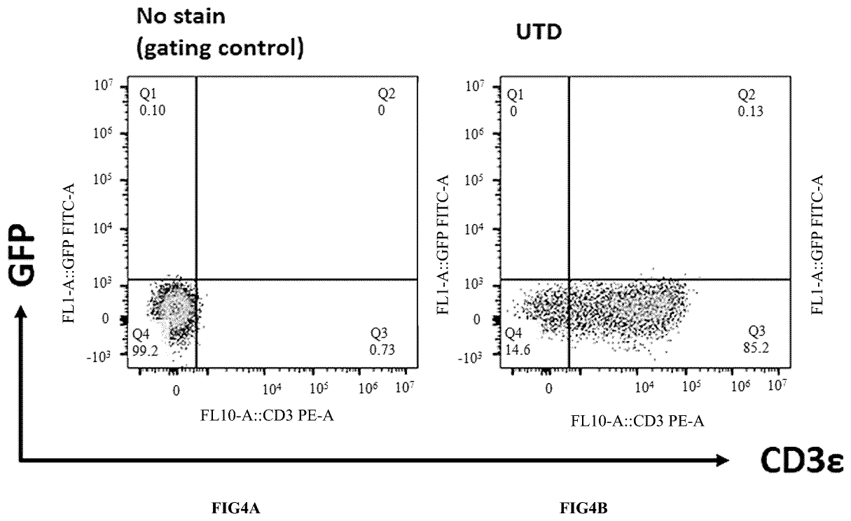

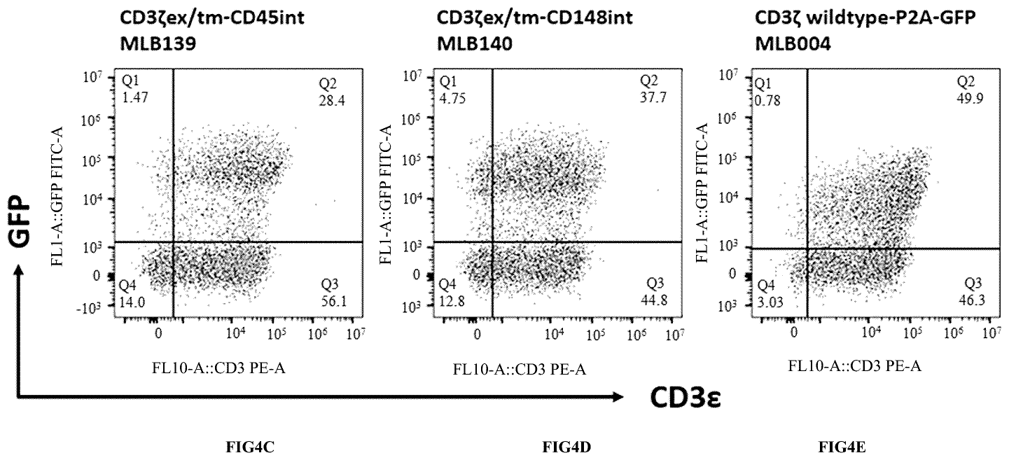

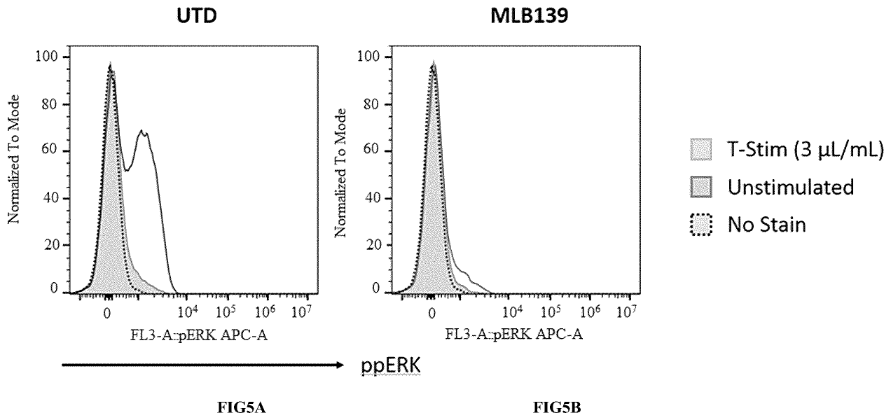

As this allogeneic system does not cause downregulation or loss of TCR expression, construct integration was validated through joint expression of GFP on the chimeric phosphatase construct. FIG. 4A shows GFP and CD3 expression levels in wildtype Jurkat control cell population without staining with fluorescently-conjugated CD3 primary antibody (BioLegend, San Diego, CA) for CD3 expression (i.e. “No stain”). FIG. 4B shows GFP and CD3 expression levels in wildtype Jurkat cell population without transducing the MLB139, MLB140 or wildtype CD3ζ construct, but staining with fluorescently-conjugated CD3 primary antibody for CD3 expression. FIGS. 4C-4E show GFP and CD3 expression levels in wildtype Jurkat cell population transducing the MLB139, MLB140 or wildtype CD3ζ construct, and staining with fluorescently-conjugated CD3 primary antibody for CD3 expression. A comparison of the expression profiles for the chimeric CD3ζ-CD45 fusion protein and CD3ζ-CD148 fusion protein and wildtype CD3ζ in FIGS. 4C-4E showed comparatively high levels of expression of both constructs MLB139 and MLB140. Indeed, as with overexpression of the wildtype CD3 subunit in MLB004, we saw TCR stabilization at the cell surface.

3. ERK Phosphorylation in Jurkat T Cells Transfected with Construct MLB139

One of the earliest readouts of TCR triggering is activation of the MAP Kinase signaling cascade. Double phosphorylation of ERK1/2 on both its tyrosine and threonine residues is a reliable readout of MAPK pathway activation. Moreover, ERK phosphorylation is proposed to be a digital readout of T cell activity.

Jurkat T cells were divided into two groups, 1) Jurkat T cells not transduced to express the MLB139 (i.e. UTD group); 2) Jurkat T cells transduced to express the chimeric phosphatase constructs MLB139 (i.e. MLB 139 group). The UTD group (FIG. 5A) and the MLB 139 group (FIG. 5B) were divided into stimulated, unstimulated, and unstained batches.

The stimulated batch (grey line marked area) was stimulated with 3 μL/mL of CD3/CD28 ImmunoCult T cell Activator (StemCell Technologies, Cambridge, MA) for ˜20 minutes at 37° C., 5% CO₂ and then fixed and permeabilized for intracellular staining using the True-Phos kit (BioLegend, San Diego, CA); then the stimulated Jurkat T Cells were stained for ERK1/2 expression using a primary AF647-conjugated antibody (BioLegend, San Diego, CA).

The unstimulated batch (filled area) was left unstimulated but stained with primary AF647-conjugated antibody and used as a baseline control for ERK phosphorylation.

The unstained batch (dotted line marked area) was not stimulated with the T cell Activator nor stained with the primary AF647-conjugated antibody and was used as a flow cytometry gating control.

ERK phosphorylation was then determined by flow cytometry using a BD CytoFlex. Jurkat T Cells were gated on GFP expression to determine which cells were positive and negative for MLB139. Untransduced Jurkat T cells negative for MLB139 (GFP−), showed an increase in ERK1/2 phosphorylation following TCR stimulation (FIG. 5A), whereas Jurkat T cells expressing MLB139 (GFP+) showed no increase in ERK1/2 phosphorylation following TCR stimulation (FIG. 5B), which means that the expression of the chimeric phosphatase completely abolished TCR-driven activation of the MAPK signaling pathway. These results indicate that expression of the chimeric phosphatase MLB139 in T cells is sufficient to eliminate the upstream TCR signaling response.

### Example 3

### Expression of Chimeric Phosphatases Prevents Jurkat T Cell Activation Through the TCR.

Jurkat T cells were electroporated with a PiggyBac transposon construct bearing MLB140 or MLB139 and Hyperactive PiggyBac transposase mRNA. Expression of the chimeric phosphatase MLB140 or MLB139 was inferred by joint GFP expression in the transduced Jurkat population (i.e. transduced Jurkat cells) (FIG. 4C-4D). CD69 is an activation marker that is upregulated within hours of T cell activation.

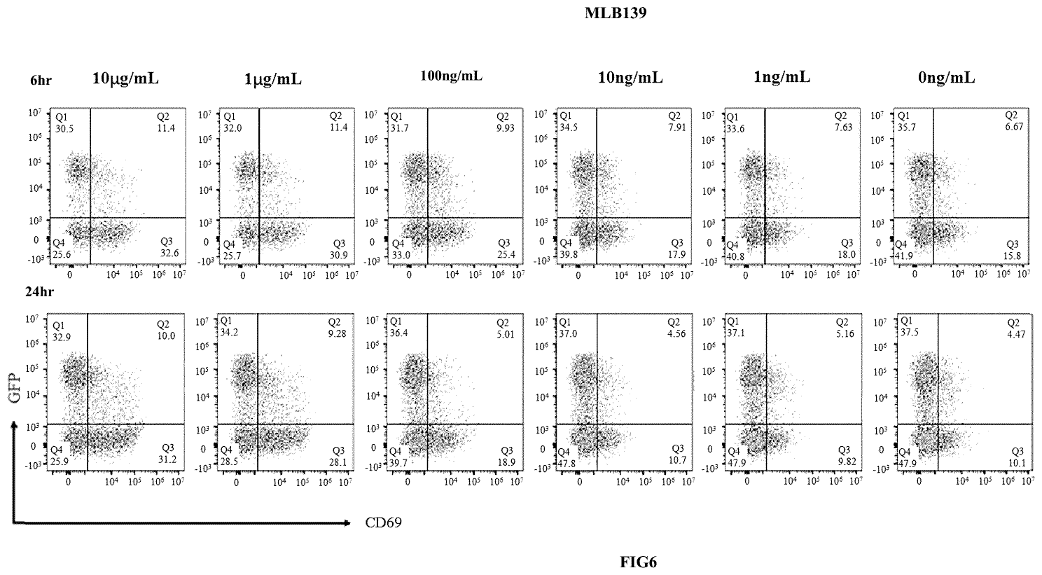

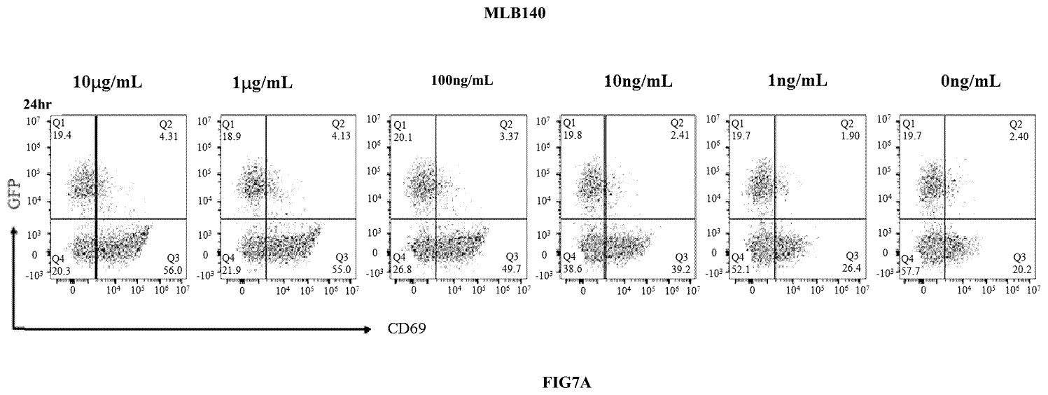

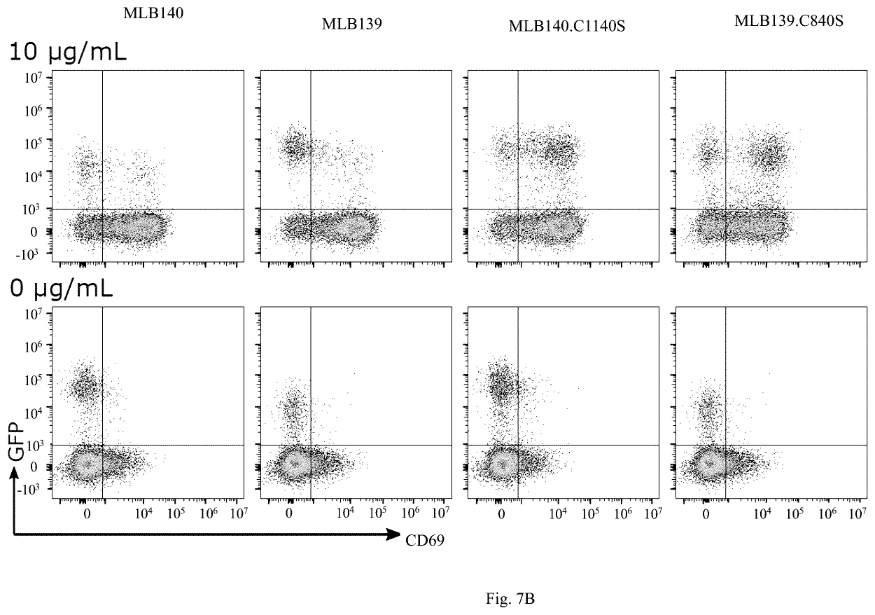

To test the effect of our chimeric phosphatases MLB140 or MLB139 on T cell activation, a titration assay was set up using plate-bound LEAF purified OKT3 antibody (an activator for T cell activation) (BioLegend, San Diego, CA). Each well of a 96-well plate was coated with either: 10 μg/mL, 1 μg/mL, 100 ng/mL, 10 ng/mL, 1 ng/mL, or 0 ng/mL LEAF purified OKT3 antibody (BioLegend, San Diego, CA). Each well was then seeded with ˜5×10⁴ transduced Jurkat cells and incubated at 37° C., 5% CO₂ for ˜6 hours or ˜24 hours. Following incubation, CD69 expression was evaluated using a PE-conjugated anti-CD69 primary antibody (BioLegend, San Diego, CA). Strikingly, CD69 upregulation was almost entirely attenuated in Jurkat cells expressing either MLB139 (FIG. 6) or MLB140 (FIG. 7A). In FIG. 6 and FIG. 7A, GFP expression was used to gate the transduced and untransduced populations (i.e. untransduced cells). In FIG. 6, in the untransduced population (lower right), we saw an increase in CD69 upregulation with increasing OKT3 concentration at both 6 hours and 24 hours; in the transduced population (upper right), there was greatly reduced CD69 upregulation even up to the highest OKT3 antibody concentration at both 6 hours and 24 hours; and in FIG. 7A, in the untransduced population (lower right), we saw an increase in CD69 upregulation with increasing OKT3 concentration at 24 hours; in the transduced population (upper right), no CD69 upregulation was observed, even at the highest OKT3 antibody concentration. These data, when combined with our previous observations on ERK1/2 phosphorylation, indicate that expression of our chimeric phosphatases is an effective means of preventing T cell activation via the TCR. FIG. 7B shows TCR signal inhibition is dependent on the catalytic activity of the phosphatase domain. CD69 expression was evaluated relative to two catalytically inactive mutant chimeric phosphatase constructs, MLB140.C1140S and MLB139.C840S. Again, we used the catalytically inactive MLB140.C1140S and MLB139.C840S phosphatase mutants as controls for our chimeric phosphatases. Both MLB140 and MLB139 strongly inhibited T cell activation following TCR stimulation, whereas Jurkat cells expressing MLB140.C1140S and MLB139.C840S still showed upregulation of CD69 in response to OKT3 stimulation.

### Example 4

CD148-Based Chimeric Phosphatase MLB140 Prevents Primary Donor-Derived T Cell Activation More Efficiently than CD45-Based Chimeric Phosphatase MLB139

### 1. Expression of the Chimeric Phosphatase Constructs MLB139 and MLB140 in Primary Donor-Derived T Cells

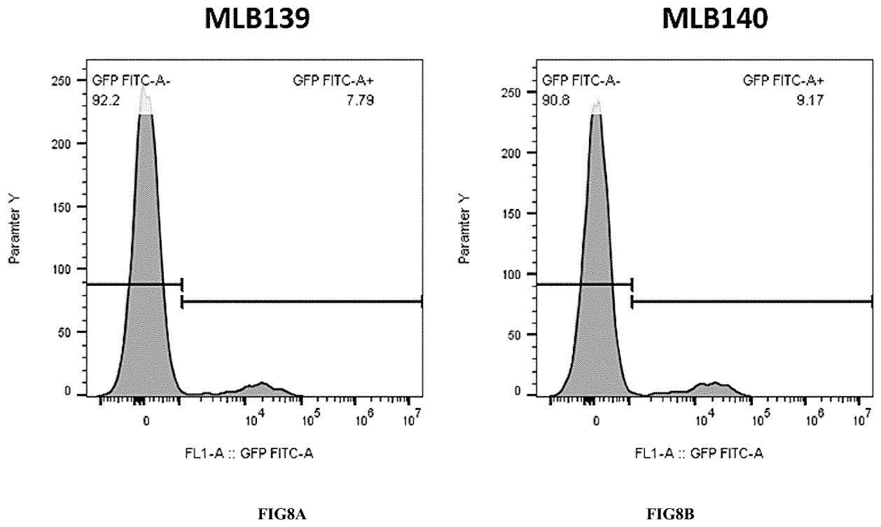

Peripheral Blood Mononuclear Cells (PBMCs) were purified by Ficoll-Paque separation and T cells were isolated via CD3 negative selection by magnetic bead separation. T cells were then stimulated using CD3/CD28 ImmunoCult T cell Activator (StemCell Technologies, Cambridge, MA) for 3 days in the presence of IL-2, IL-7, and IL-15 (PeproTech). Following stimulation, activated T cells were electroporated with PiggyBac transposon vector expressing either MLB139 or MLB140 and Hyperactive PiggyBac transposase mRNA. Expression of the chimeric phosphatase constructs was inferred via joint expression of GFP (FIG. 8A-8B). FIG. 8A shows a representative histogram of MLB139-driven GFP expression in donor-derived T cells. FIG. 8B shows a representative histogram of MLB140-driven GFP expression in donor-derived T cells.

### 2. Efficacy of Constructs MLB139 and MLB140 in Primary Donor-Derived Cells

To test the efficacy of constructs MLB139 and MLB140 in primary donor-derived T cells, T cells stably expressing the chimeric phosphatase constructs (either MLB139 or MLB140) were re-stimulated with various concentrations (10 μL/mL, 2.5 μL/mL, 0.6 μL/mL 0.625 μL/mL, or 0 μL/mL) of CD3/CD28/CD2 ImmunoCult T cell Activator (StemCell Technologies, Cambridge, MA).

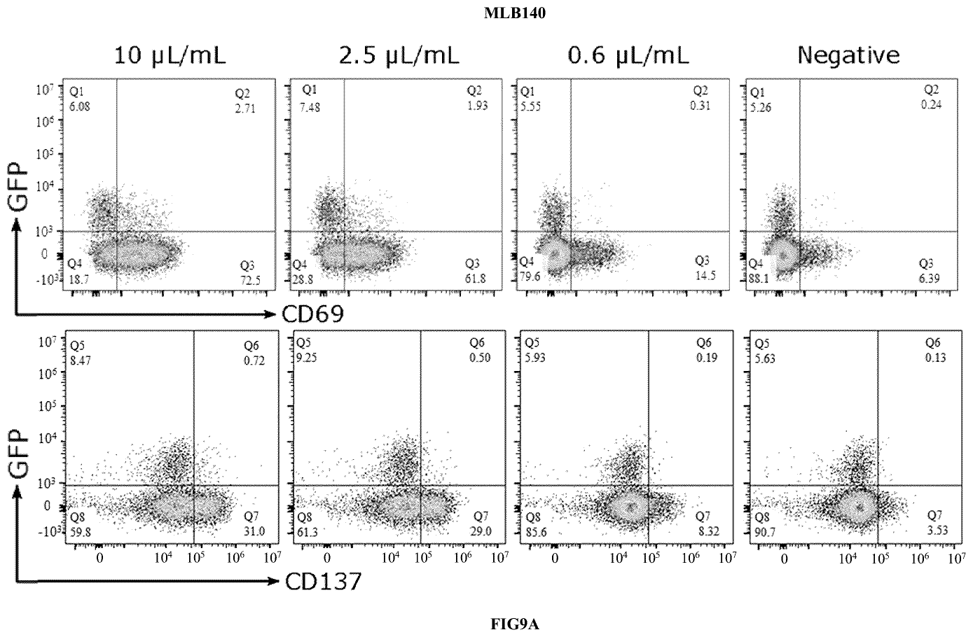

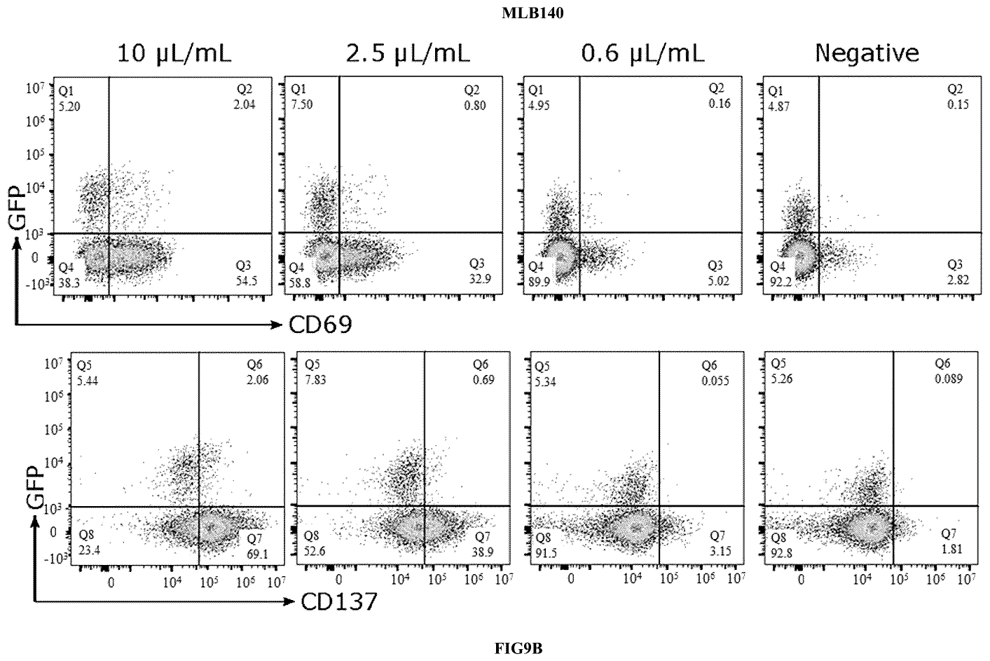

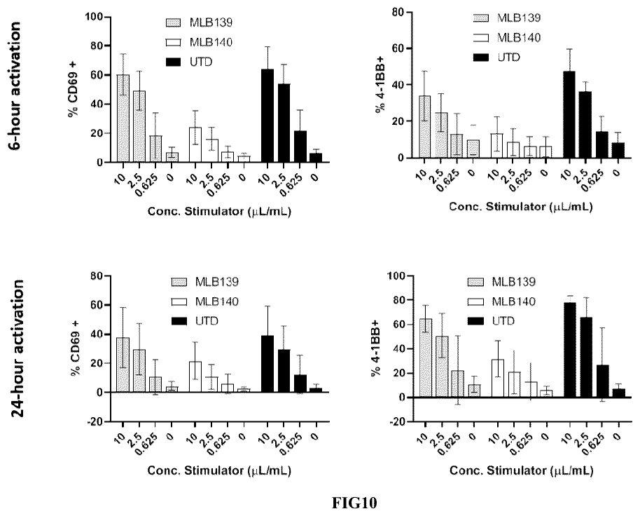

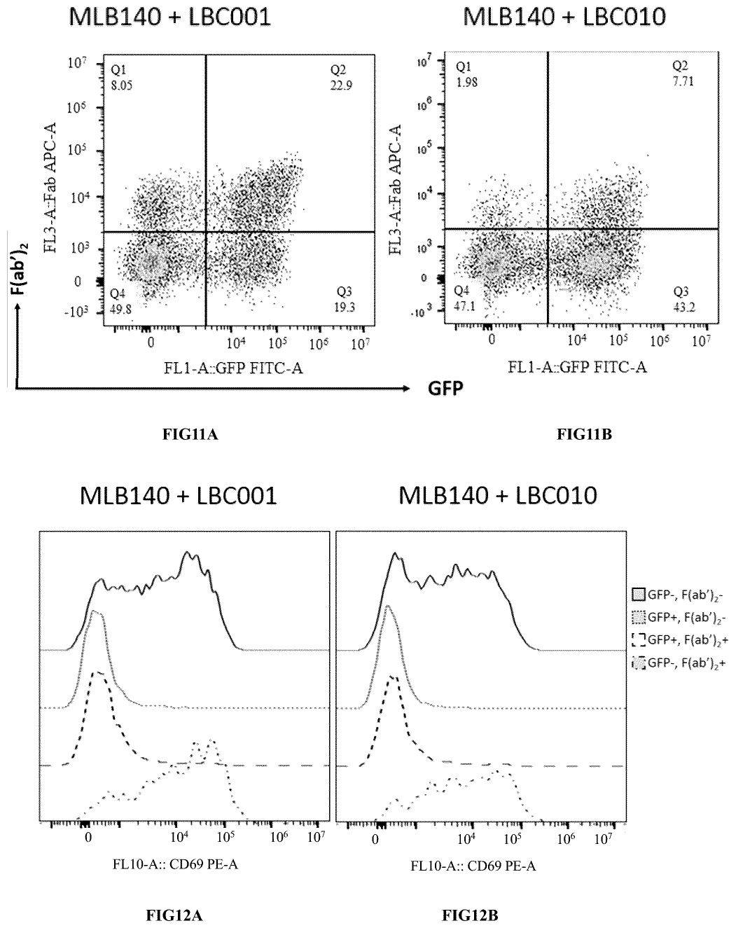

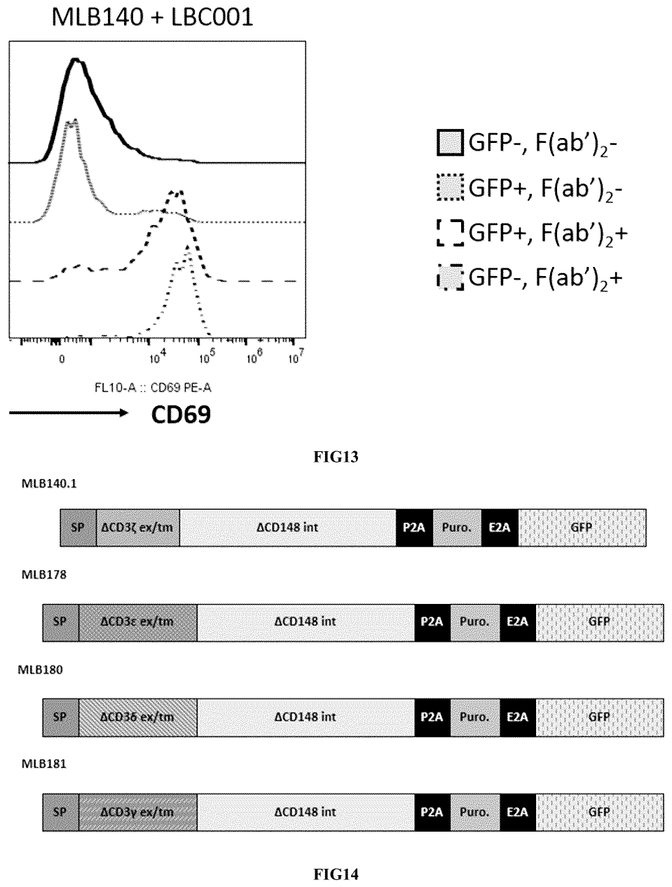

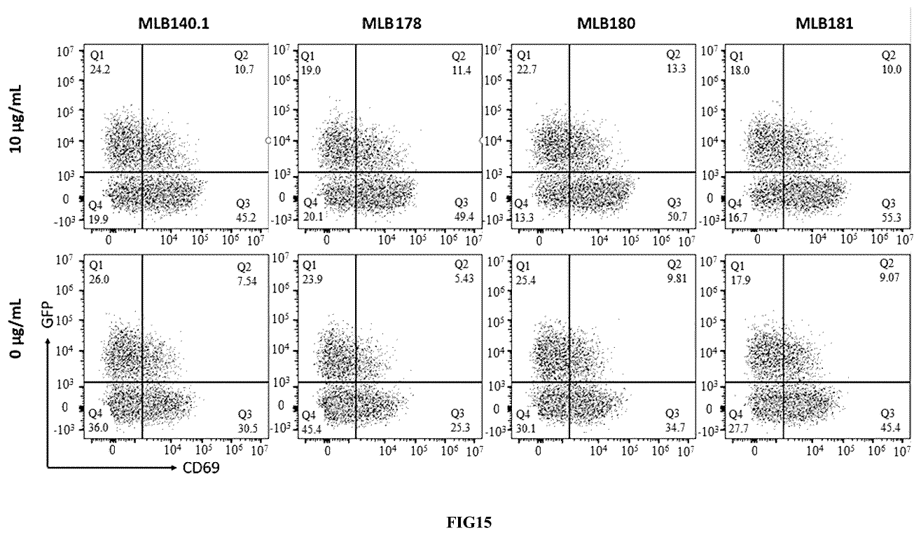

Expression of CD69 and CD137 (4-1BB) in primary donor-derived T cells transduced with MLB140 was evaluated at 6 hours post-stimulation by flow cytometry using PE-conjugated anti-CD69 primary antibody (BioLegend, San Diego, CA) and PE-conjugated anti-CD137 primary antibody (BioLegend, San Diego, CA). FIGS. 9A-B shows CD69 and CD137 expression in representative donor T cells transduced to express MLB140. These results indicated that expression of MLB140 was sufficient to attenuate T cell activation downstream of the TCR.

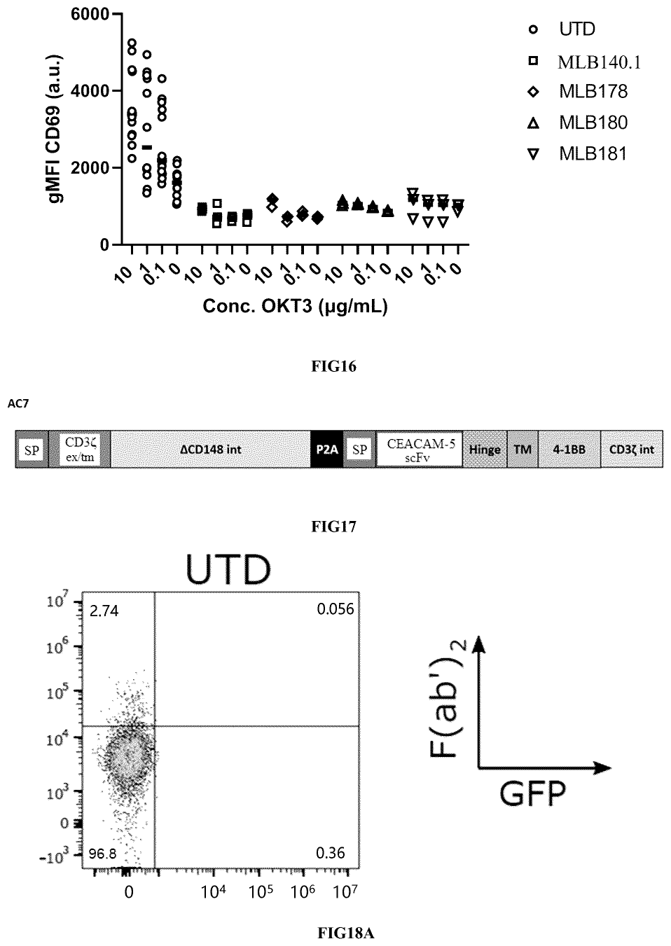

FIG. 10 shows that MLB140 prevents primary T cell activation more efficiently than MLB139 across multiple donors. Trends across all of the donors tested (n=3) were comparable and showed that construct MLB140 was effective at preventing T cell activation, as seen by the reduced fraction of both CD69 and CD137 (4-1BB) positive cells at all activator concentrations (10 μL/mL, 2.5 μL/mL, 0.625 μL/mL) at both 6 hours and 24 hours post-stimulation; MLB139 only shows reduced fractions of CD69 at activator concentrations 0.625 μL/mL at both 6 hours and 24 hours post-stimulation, and reduced fractions of CD137 (4-1BB) at activator concentrations 10 μL/mL, 2.5 μL/mL, and 0.625 μL/mL at both 6 hours and 24 hours post-stimulation.

### Example 5 CD148-Based Chimeric Phosphatase Prevents TCR-Specific Stimulation when Co-Expressed with CAR Constructs

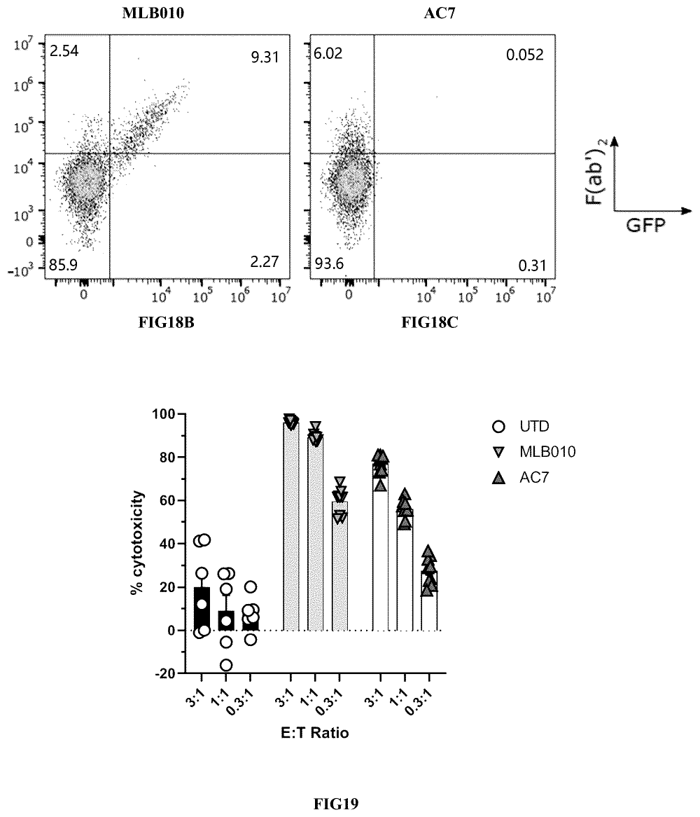

Jurkat T cells were co-transduced with MLB140 and either a CEACAM5-specific (LBC001) or CLDN18.2-specific (LBC010) CAR construct. MLB140 and LBC001, or MLB140 and LBC010 integration was mediated via co-electroporation of Hyperactive PiggyBac Transposase mRNA. MLB140 integration was inferred by co-expression of GFP on the chimeric phosphatase vector while CAR expression was detected by staining with fluorescently-conjugated anti-F(ab′)₂ antibody (Jackson ImmunoResearch). We were able to gate the cells into four distinct populations based on GFP expression and CAR expression (FIG. 11A-FIG. 11B). FIG. 11A shows gating of Jurkats doubly transduced with MLB140 and LBC001. The GFP^{Lo}, F(ab′)₂^{Lo} population is untransduced cell (lower left). The GFP^{Lo}, F(ab′)₂^{High} population is LBC001 single-positive cells (upper left)). The GFP^{High}, F(ab′)₂^{Lo} population is MLB140 single-positive (lower right). GFP^{High}, F(ab′)₂^{High} population is MLB140/LBC001 double-positive (upper right). FIG. 11B shows gating of Jurkats doubly transduced with MLB140 and LBC010. The GFP^{Lo}, F(ab′)₂^{Lo} population is untransduced cell (lower left). The GFP^{Lo}, F(ab′)₂^{High} population is LBC010 single-positive cells (upper left). The GFP^{High}, F(ab′)₂^{Lo} population is MLB140 single-positive (lower right). GFP^{High}, F(ab′)₂^{High} population is MLB140/LBC0010 double-positive (upper right).

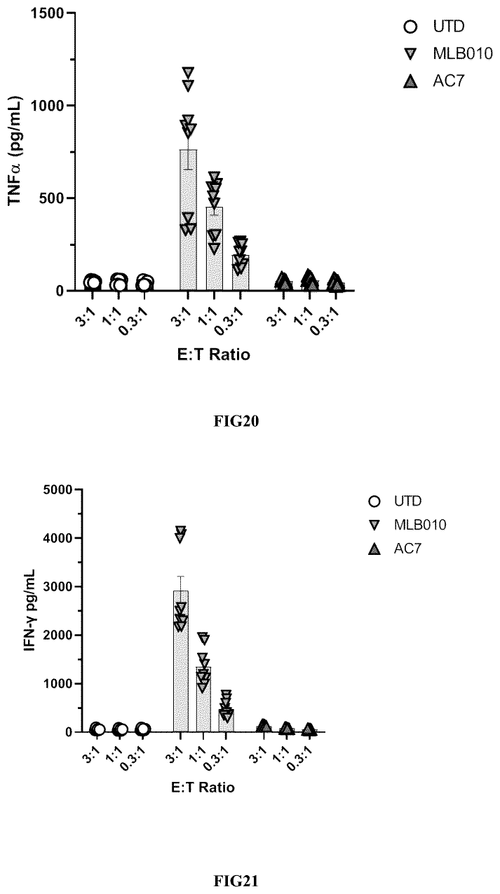

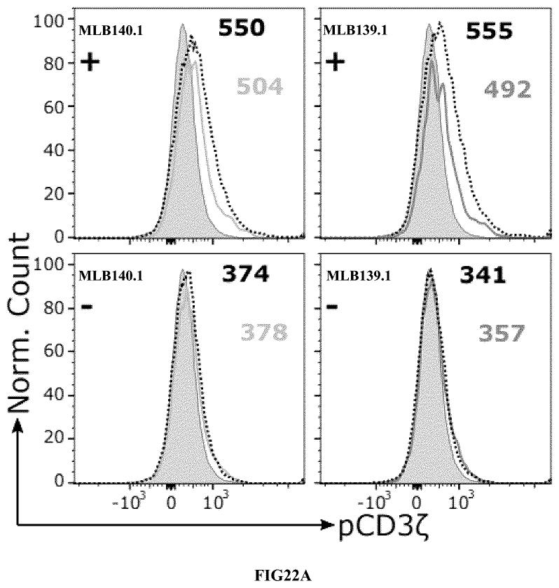

To test if the chimeric phosphatase was able to prevent TCR-driven activation (i.e. TCR signaling) when co-expressed with a CAR construct, 96-well plates were coated with LEAF purified OKT3 antibody (BioLegend, San Diego, CA) at 10.0 μg/mL, for 2 hours at 37° C., 5% CO₂. Wells were washed with PBS and then ˜5×10⁴ Jurkat T cells were seeded in 150 μL of RPMI per condition. Samples were incubated overnight and then stained with fluorescently-conjugated anti-CD69 (BioLegend) and anti-F(ab′)₂ antibodies (Jackson ImmunoResearch). Subdivision on the Jurkats on CAR and GFP expression indicated that all of the cells that were GFP-positive were not activated by OKT3 stimulation (as determined by CD69 upregulation) (the third line and the second line from top to bottom in FIG. 12A-FIG. 12B). On the other hand, Jurkat cells that were GFP-negative were activated by OKT3 stimulation regardless of CAR expression (the topmost line and bottom line in FIG. 12A-FIG. 12B). These results indicate that CAR T cells retain their ability to be activated via the TCR by the OKT3 antibody (the bottom line in FIG. 12A-FIG. 12B) and that co-expression of a chimeric phosphatase is able to prevent TCR-derived activation signals when simultaneously expressed as part of a CAR system (the third line in FIG. 12A-FIG. 12B).

### Example 6 CD148-Based Chimeric Phosphatase Specifically Ablates TCR Activation Signals while Allowing for CAR-Mediated Activation of T Cells

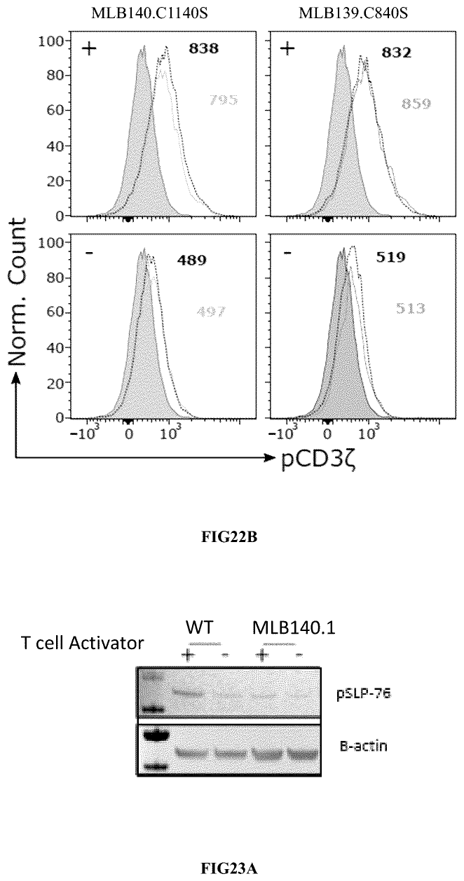

Jurkat T cells co-transduced with MLB140 and LBC001 were incubated overnight with LoVo cells endogenously expressing CEACAM-5 target antigen. Cells were cultured at a ratio of 1:2, Jurkat-to-LoVo in 150 μL of RPMI. Following overnight co-culture of the two cell lines, flow cytometry was performed to evaluate Jurkat cell activation. Cells were stained with PE/Cy7-conjugated anti-CD3 (BioLegend, San Diego, CA) to specifically gate on T cells, PE-conjugated anti-CD69 primary antibody (BioLegend, San Diego, CA) to evaluate T cell activation, and AF647-conjugated anti-F(ab′)₂ to evaluate CAR expression. Once the CD3+ T cell population had been defined, the Jurkat cells were subgated on F(ab′)₂ and GFP expression, which denote CAR and MLB140 expression, respectively. In this assay, we showed that there was no upregulation of CD69 in the untransduced population (the topmost line) and the MLB140 single-positive (GFP+) population (the second line from top to bottom). Cells expressing the CAR alone (F(ab′)₂+) (the bottom line) or the CAR co-expressed with MLB140 (GFP+, F(ab′)₂+) (the third line from top to bottom) showed strong CD69 upregulation in response to co-culture with LoVo cells (FIG. 13). These data, taken together with the OKT3 stimulation assay, indicate that chimeric phosphatase activity is specific to the TCR and does not adversely affect CAR signaling.

### Example 7 CD3ε-CD148, CD3δ-CD148 and CD3γ-CD148 Chimeric Fusion Proteins (MLB178, MLB180, and MLB181) Inhibit T Cell Activation

To test the efficacy of the CD148 fusion proteins based on CD3δ, CD3δ, and CD3γ described herein, we performed an OKT3 stimulation assay. Jurkat cells were transduced via electroporation to express MLB178, MLB180, or MLB181. Jurkat cells were also transduced to express MLB140.1 (CD148 fusion proteins based on CD3). Tissue culture plates were coated with varying concentrations (10 μg/mL, 1 μg/mL, 0.1 μg/mL, or 0 μg/mL) of OKT3 antibody (BioLegend, San Diego, CA) overnight at 4° C.

Wells were then washed once with PBS and Jurkat cells were seeded into the wells. Jurkat cells were incubated overnight at 37° C. and then stained for CD69 expression and analyzed by flow cytometry to assay the extent of T cell activation. The CD69 expression levels were evaluated using a PE-conjugated anti-CD69 primary antibody (BioLegend, San Diego, CA) (FIG. 15). GFP expression in FIG. 15 was used to gate the transduced and untransduced populations. In the untransduced population, we saw an increase in CD69 upregulation with increasing OKT3 concentration (lower right of each panel). In the transduced population there was greatly reduced CD69 upregulation even up to the higher OKT3 antibody concentration (upper right of each panel). In summary, we saw that all of the constructs tested (MLB140.1, MLB178, MLB180 and MLB181) in this assay were able to inhibit T cell activation as was observed in the decreased fraction of CD69 positive cells in the GFP positive population.

We further analyzed the extent of inhibition by measuring the gMFI of CD69 expression in the GFP positive and GFP negative populations. We found that there was a correlation between CD69 expression and OKT3 concentration in the GFP negative population, as would be expected (FIG. 16). We also witnessed a correlation between CD69 expression and OKT3 concentration in the GFP positive populations, i.e. those expressing the phosphatase constructs (MLB140.1, MLB178, MLB180, and MLB181); however, the extent of T cell activation was considerably lower than in the GFP negative populations. There were no significant differences in inhibition efficiency between the phosphatase-expressing constructs tested, indicating the potent catalytic activity of the common CD148 phosphatase domain.

### Example 8 Antigen-Specific Cell Lysis by Primary T Cells Expressing MLB140′ and CEACAM-5 CAR Co-Expression Vector

To improve electroporation efficiency in primary cells, we designed an expression vector jointly expressing both MLB140′ (that is, SP-CD3ζ ex/tm-CD148 int-P2A, or MLB140 with no GFP) and a CEACAM-5 specific CAR (FIG. 17), i.e. the AC7 construct. The structure of this AC7 construct was as follows: at the 5′ end of the construct we used the CD3ζ signal peptide followed by the CD3ζ extracellular domain and CD3 transmembrane domain. This was fused directly to the CD148 intracellular phosphatase domain to form the PhosphoTAC component of the cassette, that is, construct MLB140′ was expressed at the 5′ end of the construct. At the 3′ end of the PhosphoTAC sequence, a P2A sequence was added to drive joint expression of the CAR construct. For the CAR component of the construct, we used the CD8α signal peptide, a CEACAM-5 specific scFv, the CD8α hinge and transmembrane sequences, the 4-1BB coreceptor signaling domain, and the CD3ζ intracellular signaling domain; that is, CEACAM-5 specific CAR (i.e. CD8α signal peptide -MN14op scFv-CD8α hinge-CD8αTM-4-1BB-CD3ζ int) was expressed at the 3′ end of the construct.

To test the efficacy of this PhosphoTAC/CAR joint expression vector, we activated primary T cells derived from a healthy donor with T cell activator (STEMCELL Technologies) and then transduced them with AC7 or the parental, non-PhosphoTAC CAR MLB010 3 days post-stimulation via electroporation. Integration was monitored via CAR expression. At day 9 post electroporation, the fraction of CAR positive cells was determined by flow cytometry (FIG. 18A-FIG. 18C). FIG. 18A shows GFP and F(ab′)₂ expression levels in a Primary T cell population without transduction with MLB010 CAR or AC7 (i.e. UTD); FIG. 18B shows gating on GFP and F(ab′)₂ expression of MLB010 CAR-transduced Primary T cells. FIG. 18C shows gating on GFP and F(ab′)₂ expression of AC7-transduced primary T cells.

CAR T cells were then used in a cytotoxicity assay to compare the killing efficiency and cytokine expression of CAR T cells co-expressing the MLB140′ PhosphoTAC fusion protein and MLB010 CAR (that is, cells transduced with AC7), and the parental CAR control cells MLB010 CAR (that is, cells transduced with MLB010 CAR). CAR T cells were incubated with Luciferase-expressing LoVo cells at the indicated E:T ratios (3:1, 1:1, or 0.3:1) for 24 hours and then cytotoxicity was determined using NeoLite luciferase substrate (Promega). Percent killing was calculated as the decrease in bioluminescence in the treatment groups relative to the untreated target control cells. We observed that there was antigen-specific killing by CAR T cells expressing construct AC7, but that the killing efficiency was lower than T cells expressing only the parental CEACAM-5 specific CAR, MLB010 (FIG. 19).

Supernatant was harvested from the cytotoxicity assay samples and screened for expression of TNFα and IFNγ. The levels of both TNFα (FIG. 20) and IFNγ (FIG. 21) secreted from CAR T cells expressing AC7 were found to be dramatically lower than the corresponding levels secreted from CAR T cells expressing only the parental CAR (MLB010). Indeed, in both cases we were unable to detect cytokine secretion from CAR T cells expressing the MLB140′ PhosphoTAC-MLB010 CAR construct AC7.

### Example 9 Phospho-Flow Cytometry for Both MLB139.1 and MLB140.1 for CD3zeta Phosphorylation

Jurkat T cells transduced to express MLB139.1 and MLB140.1 were either stimulated with 20 μL of T cell Activator (STEMCELL TECHNOLOGIES) (the upper left and upper right histograms) or left unstimulated (the lower left and lower right histograms). In this example, MLB139.1 is SP-CD3ζ ex/tm-Δ CD45 int-P2A-Puro.-E2A-GFP; MLB140.1 is SP-CD3ζ ex/tm-Δ CD148 int-P2A-Puro.-E2A-GFP.

Jurkat T Cells were harvested after 10 minutes, then immediately fixed, permabilized and stained for CD3 phosphorylation using anti-pY83 CD3ζ antibody. Phospho-CD3ζ labeling was detected using an AF647-conjugated Goat anti-mouse antibody. The shaded histograms represent the secondary-only control cells to determine the background signal. The solid gray, unshaded histograms show pCD3 staining in the transduced population. The unshaded, dashed black histograms show pCD3 staining in the untransduced populations. In FIG. 22A, the stimulated Jurkat T cells (the upper left and upper right histograms) showed decreased CD3zeta phosphorylation in the transduced populations (solid gray line marked area) compared to the untransduced populations (dashed black line marked area), whereas unstimulated Jurkat T cells (the lower left and lower right histograms) showed no difference in CD3zeta phosphorylation between the transduced populations and the untransduced populations. The result of FIG. 22A shows that both constructs MLB140.1 and MLB139.1 are able to inhibit TCR signaling.

Jurkat cells were also transduced to express catalytically inactive chimeric phosphatases MLB140.C1140S and MLB139.C840S, wherein the differences between MLB140 and MLB140.C1140S, and between MLB139 and MLB139.C840S only lie in the sequence of intracellular phosphatase domain of CD148 or CD45; the intracellular phosphatase domain of CD148 in MLB140.C1140S is CD148.C1140S, the intracellular phosphatase domain of CD45 in MLB139.C840S is CD45.C840S, and the sequences of CD148.C1140S and CD45.C840S are shown in Table 1. Jurkat cells were stimulated for 10 minutes at 37° C. using 20 μL/mL of T cell Activator, then assayed for CD3 phosphorylation by intracellular flow cytometry. Cells were gated on GFP expression to identify cells expressing MLB140.C1140S (solid gray line) or MLB139.C840S (solid gray line), or untransduced control cells (dashed black line). Stimulated cells are shown in the top two histogram plots (denoted by ‘+’) and unstimulated controls are shown in the bottom two histograms (denoted by ‘−’). Fluorescence intensity was determined relative to Jurkat cells stained with secondary antibody only (shaded histogram). From FIG. 22B, it can be seen that both MLB140.C1140S or MLB139.C840S showed no difference in CD3zeta phosphorylation between the transduced populations and the untransduced populations, and that CD3ζ dephosphorylation is dependent on the catalytic activity of the specific chimeric phosphatases used.

### Example 10 Phospho-Western Blot for SLP76

Jurkat T cells were transduced to express MLB140.1 and then enriched by puromycin selection to approximately 95% purity.

Purified Jurkat T cells were allowed to rest for 48 hours following puromycin selection in puromycin-free media and then either stimulated for 2 hours with 20 μL/mL of T cell activator, or left unstimulated.

Jurkat T Cells were lysed in NP-40 buffer supplemented with protease inhibitor cocktail (Roche), PMSF, and NaVi (Millipore Sigma). Jurkat T Cell lysates were run on a 4-12% gradient gel and transferred onto a PVDF membrane. The membrane was then blocked in 2.5% Milk TBST and labeled overnight with mouse anti-human R actin (Cell Signaling Technologies) and rabbit anti-human pSLP76 (Cell Signaling Technologies).

The following day, R actin and pSLP-76 expression were detected using AF647-conjugated goat anti-mouse IgG (Cell Signaling Technologies) and DyLight 800-conjugated goat anti-rabbit IgG (Invitrogen) antibodies. The stimulated Jurkat T cells (+) showed decreased pSLP-76 phosphorylation in the populations transduced with MLB140.1 compared to the untransduced wild type (WT), whereas unstimulated Jurkat T cells (−) showed no difference in pSLP-76 expression between the transduced populations and the untransduced populations. FIG. 23A shows that MLB140.1 can inhibit signaling downstream of the TCR. FIG. 23B shows pSLP-76 fluorescence intensity was quantified relative to the band intensity of the R-actin loading control.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of these various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A fusion protein comprising: a dephosphorylation protein and a linking protein, wherein
the dephosphorylation protein comprises one or more subunits or structural domains of phosphatases or variants thereof, and
the linking protein comprises a transmembrane domain of one or more subunits of membrane-bound protein (MBP) of a T cell, or the linking protein comprises an extracellular domain and a transmembrane domain of one or more subunits of membrane-bound protein (MBP) of a T cell;
more preferably, the MBP comprises one or more of TCR or CD3; and more preferably, the MBP is a mammalian origin MBP, more preferably a human MBP;
more preferably, the extracellular domain is derived from an extracellular domain of one or more of CD3ζ, CD3γ, CD3δ, CD3ε, TCRα or TCRβ;
more preferably, the transmembrane domain is derived from a transmembrane domain of one or more of CD3ζ, CD3γ, CD3δ, CD3ε, TCRα or TCRβ.

2. The fusion protein of claim 1, wherein the phosphatases are receptor-like protein tyrosine phosphatases; preferably, the dephosphorylation protein comprises an intracellular phosphatase domain of the receptor-like protein tyrosine phosphatases or a variant thereof; more preferably, the receptor-like protein tyrosine phosphatases comprise CD45 or CD148.

3. The fusion protein of claim 2, wherein, the fusion protein comprises from N-terminal to C-terminal:
(1) the linking protein; and (2) the dephosphorylation protein;
preferably, (1) a transmembrane domain of one or more subunits of membrane-bound protein (MBP) of a T cell; and (2) the dephosphorylation protein;
preferably, (1) an extracellular domain, a transmembrane domain of one or more subunits of membrane-bound protein (MBP) of a T cell; and (2) the dephosphorylation protein;
more preferably, (1) the transmembrane domain of one or more of CD3ζ, CD3γ, CD3δ, CD3ε, TCRα or TCRβ or variants thereof, and (2) the intracellular phosphatase domain of CD45 or CD148 or a variant thereof; or
more preferably, (1) the extracellular domain of one or more of CD3ζ, CD3γ, CD3δ, CD3ε, TCRα or TCRβ or variants thereof, the transmembrane domain of one or more of CD3ζ, CD3γ, CD3δ, CD3ε, TCRα or TCRβ or variants thereof, and (2) the intracellular phosphatase domain of CD45 or CD148 or a variant thereof.

4. The fusion protein of claim 14, wherein
the fusion protein is capable of mediating dephosphorylation of proteins that regulate T cell activation, and the proteins that regulate T cell activation comprise one or more subunits or structural domains of a protein selected from one or more of a membrane-bound protein (MBP), a kinase, or a scaffold protein of the T cell;
preferably, the proteins that regulate T cell activation comprise one or more subunits or structural domains of TCR or CD3; more preferably, the TCR or CD3 is a mammalian origin TCR or CD3, more preferably a human TCR or CD3; and more preferably an endogenous TCR or CD3; more preferably, the proteins that regulate T cell activation comprise ITAM domains of the membrane-bound protein (MBP), preferably ITAM domains of CD3, more preferably ITAM domains of CD3ζ;
preferably, the kinase comprises ZAP70; and more preferably, the kinase is a mammalian origin kinase, more preferably, a human kinase; or
preferably, the scaffold protein comprises LAT and/or SLP76; and more preferably, the scaffold protein is a mammalian origin scaffold protein, more preferably, a human scaffold protein.

5. The fusion protein of claim 3, wherein the transmembrane domain of CD3ζ comprises an amino acid sequence at least about 95%, 96%, 97%, 98%, 99% or 100% identical to the amino acid sequence shown in SEQ ID NO:3, the transmembrane domain of CD3ε comprises an amino acid sequence at least about 95%, 96%, 97%, 98%, 99% or 100% identical to the amino acid sequence shown in SEQ ID NO.:28, the transmembrane domain of CD3γ comprises an amino acid sequence at least about 95%, 96%, 97%, 98%, 99% or 100% identical to the amino acid sequence shown in SEQ ID NO.:31, the transmembrane domain of CD3δ comprises an amino acid sequence at least about 95%, 96%, 97%, 98%, 99% or 100% identical to the amino acid sequence shown in SEQ ID NO.:34;
the extracellular domain of CD3ζ comprises an amino acid sequence at least about 95%, 96%, 97%, 98%, 99% or 100% identical to the amino acid sequence shown in SEQ ID NO:2, the extracellular domain of CD3ε comprises an amino acid sequence at least about 95%, 96%, 97%, 98%, 99% or 100% identical to the amino acid sequence shown in SEQ ID NO.:27, the extracellular domain of CD3γ comprises an amino acid sequence at least about 95%, 96%, 97%, 98%, 99% or 100% identical to the amino acid sequence shown in SEQ ID NO.:30, the extracellular domain of CD3δ comprises an amino acid sequence at least about 95%, 96%, 97%, 98%, 99% or 100% identical to the amino acid sequence shown in SEQ ID NO.:33; and/or
the intracellular phosphatase domain of CD45 comprises an amino acid sequence at least about 95%, 96%, 97%, 98%, 99% or 100% identical to the amino acid sequence shown in SEQ ID NO.:4; or the intracellular phosphatase domain of CD148 comprises an amino acid sequence at least about 95%, 96%, 97%, 98%, 99% or 100% identical to the amino acid sequence shown in SEQ ID NO.:5.

6. The fusion protein of claim 3, wherein the fusion protein comprises:
a signal peptide sequence, wherein the signal peptide sequence comprises an amino acid sequence at least about 95%, 96%, 97%, 98%, 99% or 100% identical to the amino acid sequence shown in any one of SEQ ID NOs:1, 26, 29 and 32;
preferably, the CD3ζ signal peptide sequence comprises an amino acid sequence at least about 95%, 96%, 97%, 98%, 99% or 100% identical to the amino acid sequence shown in SEQ ID NO.:1, the CD3ε signal peptide sequence comprises an amino acid sequence at least about 95%, 96%, 97%, 98%, 99% or 100% identical to the amino acid sequence shown in SEQ ID NO.:26, the CD3γ signal peptide sequence comprises an amino acid sequence at least about 95%, 96%, 97%, 98%, 99% or 100% identical to the amino acid sequence shown in SEQ ID NO.:29, and the CD3δ signal peptide sequence comprises an amino acid sequence at least about 95%, 96%, 97%, 98%, 99% or 100% identical to the amino acid sequence shown in SEQ ID NO.:32.

7. A nucleic acid comprising a polynucleotide encoding a fusion protein of claim 1.

8. (canceled)

9. A vector comprising the nucleic acid of claim 7.

10. A composition comprising a nucleic acid of claim 7.

11. A composition comprising a first nucleic acid and a second nucleic acid, comprising a vector having a first nucleic acid and a second nucleic acid, or comprising a first vector having a first nucleic acid and a second vector having a second nucleic acid, wherein
(1) the first nucleic acid encodes a fusion protein of claim 1, and
(2) the second nucleic acid encodes a chimeric antigen receptor (CAR) comprising:
(a) an extracellular ligand-binding domain comprising a single chain variable fragment (scFv) specifically binding to a predetermined antigen;
(b) a transmembrane domain, preferably CD8α, CD28, 4-1BB or IL2R transmembrane domain, more preferably CD8α transmembrane domain, and
(c) a cytoplasmic segment comprising one or more signaling domains, preferably comprising a 4-1BB signaling domain and a CD3ζ signaling domain.

12. The composition of claim 11, wherein the predetermined antigen is a tumor-related antigen,
preferably, the tumor-related antigen is selected from the following group: CEA, Claudin 18.2, GPC3, Receptor tyrosine kinase-like Orphan Receptor 1 (ROR1), CD3δ, CD19, CD20, CD22, BCMA, CAIX, CD446, CD133, EGFR, EGFRvIII, EpCam, GD2, EphA2, Her1, Her2, ICAM-1, IL13Ra2, Mesothelin, MUC1, MUC16, NKG2D, PSCA, NY-ESO-1, MART-1, WT1, MAGE-A10, MAGE-A3, MAGE-A4, EBV, NKG2D, PD1, PD-L1, CD25, IL-2, and CD3ζ;
preferably, the tumor-related antigen is CEA, more preferably CEACAM5.

13. A host cell comprising a nucleic acid of claim 7; wherein
preferably, the host cell is a mammalian T cell, preferably a human T cell;
preferably, the host cell is a Jurkat cell, a primary T cell, a gamma delta T cell, or a NK T cell; and
preferably, the host cell is an allogeneic T cell.

14. A pharmaceutical composition comprising a nucleic acid of claim 7; wherein preferably, the composition further comprises one or more pharmaceutically acceptable excipients.

15. A method for dephosphorylation of proteins that regulate T cell activation through the TCR signaling pathway, wherein preferably, the proteins rely on phosphorylation as an activation mechanism for signaling in the TCR signaling pathway, preferably, the proteins are Immunoreceptor Tyrosine-based Activation Motifs (ITAMs) of a membrane-bound protein (MBP) in the TCR signaling pathway of a T cell; optionally, the MBP is an endogenous MBP, comprising introducing into a T cell the nucleic acid of claim 7; wherein
preferably, the T cell is a mammalian T cell, preferably a human T cell;
preferably, the T cell is a Jurkat cell, a primary T cell, a gamma delta T cell, or a NKT cell; and
preferably, the T cell is an allogeneic T cell.

16. A method of producing a T cell having dephosphorylated proteins that regulate T cell activation through the TCR signaling pathway, wherein preferably, the proteins rely on phosphorylation as an activation mechanism for signaling in the TCR signaling pathway, preferably, the proteins are Immunoreceptor Tyrosine-based Activation Motifs (ITAMs) of a membrane-bound protein (MBP) in the TCR signaling pathway; optionally, the MBP is an endogenous MBP, comprising introducing into a T cell the nucleic acid of claim 7; wherein
preferably, the T cell is a mammalian T cell, preferably a human T cell;
preferably, the T cell is a Jurkat cell, a primary T cell, a gamma delta T cell, or a NKT cell; and
preferably, the T cell is an allogeneic T cell.

17. A method of treating a disease, comprising administering to a subject in need thereof a therapeutically effective amount of a composition of claim 10; wherein
preferably, the host cell is a mammalian T cell, preferably a human T cell;
preferably, the host cell is a Jurkat cell, a primary T cell, a gamma delta T cell, or a NKT cell;
preferably, the host cell is an allogeneic T cell; and
preferably, the subject has reduced Graft-versus-Host Disease (GvHD).

18. A method of reducing or preventing Graft-versus-Host Disease (GvHD) in a subject associated with the administration of one or more CAR T-cells to the subject, comprising:
(1) transducing one or more CAR T-cells with a nucleic acid of claim 7 and
(2) administering the transduced CAR T-cells to the subject.

19. A host cell comprising a composition of claim 11; wherein
preferably, the host cell is a mammalian T cell, preferably a human T cell;
preferably, the host cell is a Jurkat cell, a primary T cell, a gamma delta T cell, or a NK T cell; and
preferably, the host cell is an allogeneic T cell.

20. A pharmaceutical composition comprising a composition of claim 11; wherein preferably, the composition further comprises one or more pharmaceutically acceptable excipients.

21. A method of treating a disease, comprising administering to a subject in need thereof a therapeutically effective amount of a composition of claim 11; wherein
preferably, the host cell is a mammalian T cell, preferably a human T cell;
preferably, the host cell is a Jurkat cell, a primary T cell, a gamma delta T cell, or a NKT cell;
preferably, the host cell is an allogeneic T cell; and
preferably, the subject has reduced Graft-versus-Host Disease (GvHD).


SEQUENCE LISTING (36 sequences)

SEQ ID NO: 1 - protein, 21 aa, synthetic construct
MKWKALFTAAILQAQLPITEA

SEQ ID NO: 2 - protein, 9 aa, synthetic construct
QSFGLLDPK

SEQ ID NO: 3 - protein, 21 aa, synthetic construct
LCYLLDGILFIYGVILTALFL

SEQ ID NO: 4 - protein, 708 aa, synthetic construct
YKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 5 - protein, 341 aa, synthetic construct
RKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 6 - protein, 22 aa, synthetic construct
GSGATNFSLLKQAGDVEENPGP

SEQ ID NO: 7 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 8 - protein, 1020 aa, synthetic construct
MKWKALFTAAILQAQLPITEAQSFGLLDPKLCYLLDGILFIYGVILTALFLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGSGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 9 - protein, 653 aa, synthetic construct
MKWKALFTAAILQAQLPITEAQSFGLLDPKLCYLLDGILFIYGVILTALFLRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIAGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 10 - protein, 421 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRNPQEGLYNELQKDKMAEAYSEIGMKGERRRGKGHDGLYQGLSTATKDTYDALHMQALPPRATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 11 - protein, 21 aa, synthetic construct
MALPVTALLLPLALLLHAARP

SEQ ID NO: 12 - protein, 246 aa, synthetic construct
EVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKG

SEQ ID NO: 13 - protein, 249 aa, synthetic construct
QVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSSGGGGSGGGGSGGGGSDIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIKRT

SEQ ID NO: 14 - protein, 45 aa, synthetic construct
TTTPAPRPPTPAPTIASQPLSLRPEACRPAAGGAVHTRGLDFACD

SEQ ID NO: 15 - protein, 24 aa, synthetic construct
IYIWAPLAGTCGVLLLSLVITLYC

SEQ ID NO: 16 - protein, 42 aa, synthetic construct
KRGRKKLLYIFKQPFMRPVQTTQEEDGCSCRFPEEEEGGCEL

SEQ ID NO: 17 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 490 aa, synthetic construct
MALPVTALLLPLALLLHAARPEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 493 aa, synthetic construct
MALPVTALLLPLALLLHAARPQVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSSGGGGSGGGGSGGGGSDIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 19 aa, synthetic construct
ATNFSLLKQAGDVEENPGP

SEQ ID NO: 21 - protein, 748 aa, synthetic construct
MALPVTALLLPLALLLHAARPEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 22 - protein, 19 aa, synthetic construct
ATNFSLLKQAGDVEENPGP

SEQ ID NO: 23 - protein, 239 aa, synthetic construct; identical to SEQ ID NO: 7

SEQ ID NO: 24 - protein, 23 aa, synthetic construct
GSGQCTNYALLKLAGDVESNPGP

SEQ ID NO: 25 - protein, 199 aa, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 26 - protein, 22 aa, synthetic construct
MQSGTHWRVLGLCLLSVGVWGQ

SEQ ID NO: 27 - protein, 104 aa, synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 28 - protein, 26 aa, synthetic construct
VMSVATIVIVDICITGGLLLLVYYWS

SEQ ID NO: 29 - protein, 22 aa, synthetic construct
MEQGKGLAVLILAIILLQGTLA

SEQ ID NO: 30 - protein, 94 aa, synthetic construct
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 31 - protein, 21 aa, synthetic construct
GFLFAEIVSIFVLAVGVYFIA

SEQ ID NO: 32 - protein, 21 aa, synthetic construct
MEHSTFLSGLVLATLLSQVSP

SEQ ID NO: 33 - protein, 84 aa, synthetic construct [sequence omitted]

SEQ ID NO: 34 - protein, 21 aa, synthetic construct
GIIVTDVIATLLLALGVFCFA

SEQ ID NO: 35 - protein, 708 aa, synthetic construct
YKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHSSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 36 - protein, 341 aa, synthetic construct
RKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHSSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA